(19) United States
(12) Patent Application Publication
(10) Pub. No.: US 2024/0091533 A1
(43) Pub. Date: Mar. 21, 2024

(54) MONITORING AND TREATING ATRIAL FIBRILLATION, ARRYTHMIA, AND ADDITIONAL CONDITIONS

(71) Applicant: ZIV HEALTHCARE LTD., Nes-Ziona (IL)

(72) Inventors: Amos ZIV, Rehovot (IL); Yitzhak HOLAND, Rehovot (IL)

(73) Assignee: ZIV HEALTHCARE LTD., Nes-Ziona (IL)

(21) Appl. No.: 18/272,870

(22) PCT Filed: Jan. 18, 2022
(86) PCT No.: PCT/IL2022/050074

§ 371 Date: Jul. 18, 2023

Related U.S. Application Data
(60) Provisional application No. 63/210,570, filed on Jun. 15, 2021.
(60) Provisional application No. 63/138,561, filed on Jan. 18, 2021.

(51) Int. Cl. A61N 1/36; A61N 1/04

(52) U.S. Cl. CPC A61N 1/36031; A61N 1/0404; A61N 1/36034

(57) ABSTRACT

A system comprising a control module; a neuro-stimulation unit operationally controlled by the control module and configured to generate an electrical stimulation signal at a frequency of between 40-50 Hz; and at least two electrodes configured to be positioned in simultaneous dermal contact with an inner surface of the wrist of a subject, proximately to a median nerve of the subject, wherein each of the at least two electrodes is connected to the neuro-stimulation unit for delivering the generated electrical stimulation signal from the neuro-stimulation unit to the subject, wherein the electrical stimulation signal is configured to apply a neuromodulation treatment to the subject, to reduce occurrences of an arrhythmia-related condition in the subject.

### CROSS-REFERENCE TO RELATED APPLICATIONS

This application claims the benefit of priority from U.S. Provisional Patent Application No. 63/138,561, filed Jan. 18, 2021, entitled, “MONITORING AND TREATING ATRIAL FIBRILLATION, ARRYTHMIA, AND ADDITIONAL CONDITIONS,” and from U.S. Provisional Patent Application No. 63/210,570, filed Jun. 15, 2021, entitled, “MONITORING AND TREATING ATRIAL FIBRILLATION, ARRYTHMIA, AND ADDITIONAL CONDITIONS,” the contents of both of which are hereby incorporated by reference in their entirety.

### FIELD OF EMBODIMENTS OF THE INVENTION

Some applications of the present invention generally relate to devices and methods for monitoring and treating arrythmia-related events.

### BACKGROUND

Neuromodulation is used to treat various conditions, ranging from psychiatric disorders, such as depression, to physiological conditions, such as chronic pain. In particular, neuromodulation of peripheral nerves may be used to modulate the autonomic nervous system.

Portable devices for monitoring and treating various physiological conditions allow for real-time treatment, whether in response to an acute crisis, or to effect a general state of wellness and improved mood. Recent years have seen a sharp rise in the number of consumer wearable devices designed to monitor physiological conditions, from devices designed to track vital signs, such as heart-rate or blood pressure, to recreational wearable devices, such as wearable fitness trackers.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in an embodiment, a system comprising a control module; a neuro-stimulation unit operationally controlled by the control module and configured to generate an electrical stimulation signal at a frequency of between 40-50 Hz; and at least two electrodes configured to be positioned in simultaneous dermal contact with an inner surface of the wrist of a subject, proximately to a median nerve of the subject, wherein each of the at least two electrodes is connected to the neuro-stimulation unit for delivering the generated electrical stimulation signal from the neuro-stimulation unit to the subject, wherein the electrical stimulation signal is configured to apply a neuromodulation treatment to the subject, to reduce occurrences of an arrhythmia-related condition in the subject.

There is also provided, in an embodiment, a method comprising providing a system comprising a control module, a neuro-stimulation unit operationally controlled by the control module and configured to generate an electrical stimulation signal at a frequency of between 40-50 Hz, and at least two electrodes configured to be positioned in simultaneous dermal contact with an inner surface of the wrist of a subject, proximately to a median nerve of the subject, wherein each of the at least two electrodes is connected to the neuro-stimulation unit for delivering the generated electrical stimulation signal from the neuro-stimulation unit to the subject, wherein the electrical stimulation signal is configured to apply a neuromodulation treatment to the subject, to reduce occurrences of an arrhythmia-related condition in the subject; and placing the at least two electrodes in simultaneous dermal contact with an inner surface of the wrist of the subject, such that each of the at least two electrodes is positioned along a lengthwise axis thereof, proximately, and substantially parallel to, a longitudinal axis of the median nerve.

In some embodiments, the control module is configured to operate the neuro-stimulation unit to generate the electrical stimulation signal according to a predetermined schedule, wherein the generated electrical stimulation signal is delivered from the neuro-stimulation unit via the at least two electrodes to the subject.

In some embodiments, the control module is configured to receive, as input, data that is indicative of at least one parameter selected from the groups of parameters consisting of: activity status parameters of the subject, prandial status parameters of the subject, and emotional state parameters of the subject.

In some embodiments, the control module is configured to predict, based on the data, a current or oncoming occurrence of the arrythmia-related condition with respect to the subject, and to operate the neuro-stimulation unit to generate the electrical stimulation signal based, at least in part, on the predicting, wherein the generated electrical stimulation signal is delivered from the neuro-stimulation unit via the at least two electrodes to the subject.

In some embodiments, the activity status parameters of the subject indicate that the subject is at least one of: eating, lying down, sleeping, walking, running, exercising, or driving; the prandial status parameters of the subject indicate that the subject is at least one of: hungry, currently eating, or recently ate; and the emotional state parameters of the subject indicate that the subject is at least one of: stressed, relaxed, in a positive mood, depressed, anxious, or traumatized.

In some embodiments, the control module is further configured to receive, as an additional input, data that is indicative of at least one additional parameter with respect to the subject, selected from the group consisting of: fatigue, tightness of the chest, palpitations, dizziness, fainting, headaches, shortness of breath, sensation of “emptiness” in the chest, rapid or fluttering heartbeats, skipping heart beats, pressure in the throat, coldness or chills, dehydration, blood-related parameters, anemia diagnosis, digestion-related symptoms, insomnia, and hormonal data.

In some embodiments, the predicting is based, at least in part, on correlating, in the subject, at least one of the parameters comprising the data, with an occurrence of the arrythmia-related condition, wherein the correlating is based on current and historical information associated with occurrences of the arrythmia-related condition in the subject.

In some embodiments, the control module is further configured to receive, as an additional input, a heart activity signal of the subject.

In some embodiments, the control module is further configured to process the heart activity signal to derive one or more heart activity-related parameter selected from the group consisting of: heart rate variability (HRV), heart rate recovery, heart rate reserve, premature atrial contractions (PAC), ventricular premature contractions, atrial tachycardia, RR interval, average interval between normal heart beats (AVNN), standard deviation of NN intervals (SDNN), root mean square of successive differences between normal heartbeats (rMSSD), pNN50, low frequency activity (LF), and high frequency activity (HF).

In some embodiments, the control module is further configured to detect, based on the heart activity signal, a current or oncoming occurrence of the arrythmia-related condition with respect to the subject, and to operate the neuro-stimulation unit to generate the electrical stimulation signal based, at least in part, on the detecting, wherein the generated electrical stimulation signal is delivered from the neuro-stimulation unit via the at least two electrodes to the subject.

In some embodiments, the control module is further configured to determine that the autonomous nervous system of the subject is in a sympathetic state or a parasympathetic state.

In some embodiments, when the autonomous nervous system of the subject is in a sympathetic state, the control module is configured to operate the neuro-stimulation unit to generate the electrical stimulation signal at a frequency of between 1-5 Hz.

In some embodiments, the system further comprises at least one of: an electrocardiogram (ECG) sensor, a photoplethysmogram (PPG) sensor, and an accelerometer.

In some embodiments, the system is housed, at least in part, within a wearable device configured to be worn by the subject.

In some embodiments, the wearable device comprises a wristband configured to be worn around a wrist of the subject such that the at least two electrodes are each positioned, along a lengthwise axis thereof, proximately, and substantially parallel to, a longitudinal axis of the median nerve.

In some embodiments, the arrhythmia-related condition is one or more of: atrial fibrillation (AF), premature atrial complex (PAC), premature ventricular complex (PVC), supraventricular tachycardia (SVT), atrial tachycardia (AT), atrial flutter, atrioventricular node re-entrant tachycardia (AVNRT), paroxysmal supraventricular tachycardia (PSVT), atrioventricular re-entrant tachycardia, Wolff-Parkinson-White syndrome, ventricular tachycardia (VT), torsades de pointes (TdP), long QT syndrome, heart block, and sick sinus syndrome.

In some embodiments, the control module is further configured to measure an electrical current associated with the electrical stimulation signal between the at least two electrodes, and to determine that a positioning of the at least two electrodes relative to the inner surface of the wrist of the subject is incorrect when the measured electrical current is lower than a predetermined baseline value.

In some embodiments, the predetermined baseline value is determined by measuring an electrical current associated with the electrical stimulation signal between the at least two electrodes, when the at least two electrodes are each positioned along a lengthwise axis thereof proximately, and substantially parallel, to a longitudinal axis of the median nerve.

There is further provided, in an embodiment, a system comprising a control module; a neuro-stimulation unit operationally controlled by the control module and configured to generate an electrical stimulation signal; and at least two electrodes configured to be positioned in simultaneous dermal contact with an inner surface of the wrist of a subject, proximately to a median nerve of the subject, wherein each of the at least two electrodes is connected to the neuro-stimulation unit for delivering the generated electrical stimulation signal from the neuro-stimulation unit to the subject, wherein the control module is configured to: receive, as input, a heart activity signal of the subject, detect, based on the heart activity signal, a current or oncoming occurrence of an arrythmia-related condition with respect to the subject, and operate the neuro-stimulation unit to generate the electrical stimulation signal at a frequency of between 40-50 Hz, wherein the generated electrical stimulation signal is delivered from the neuro-stimulation unit via the at least two electrodes to the subject, and wherein the electrical stimulation signal is configured to apply a neuromodulation treatment to the subject, to reduce occurrences of the arrhythmia-related condition in the subject.

There is further provided, in an embodiment, a method comprising: providing a system comprising a control module; a neuro-stimulation unit operationally controlled by the control module and configured to generate an electrical stimulation signal; and at least two electrodes configured to be positioned in simultaneous dermal contact with an inner surface of the wrist of a subject, proximately to a median nerve of the subject, wherein each of the at least two electrodes is connected to the neuro-stimulation unit for delivering the generated electrical stimulation signal from the neuro-stimulation unit to the subject, wherein the control module is configured to: receive, as input, a heart activity signal of the subject, detect, based on the heart activity signal, a current or oncoming occurrence of an arrythmia-related condition with respect to the subject, and operate the neuro-stimulation unit to generate the electrical stimulation signal at a frequency of between 40-50 Hz, wherein the generated electrical stimulation signal is delivered from the neuro-stimulation unit via the at least two electrodes to the subject, and wherein the electrical stimulation signal is configured to apply a neuromodulation treatment to the subject, to reduce occurrences of the arrhythmia-related condition in the subject; and placing the at least two electrodes in simultaneous dermal contact with an inner surface of the wrist of the subject, such that each of the at least two electrodes is positioned along a lengthwise axis thereof, proximately, and substantially parallel to, a longitudinal axis of the median nerve.

In some embodiments, the control module is configured to detect the oncoming occurrence of the arrythmia-related condition, based, at least in part, on detecting, in the heart activity signal, at least one of: premature atrial complexes (PAC), and premature ventricular complexes (PVC).

In some embodiments, the detecting is based on measuring, in the heart activity signal, a percentage increase in the one of PAC and PVC, relative to a baseline measurement in the subject.

In some embodiments, the system further comprises at least one of: an electrocardiogram (ECG) sensor, a photoplethysmogram (PPG) sensor, and an accelerometer.

In some embodiments, the control module is further configured to process the heart activity signal to derive one or more heart activity-related parameter selected from the group consisting of: heart rate variability (HRV), heart rate recovery, heart rate reserve, premature atrial contractions (PAC), ventricular premature contractions, atrial tachycardia, RR interval, average interval between normal heart beats (AVNN), standard deviation of NN intervals (SDNN), root mean square of successive differences between normal heartbeats (rMSSD), pNN50, low frequency activity (LF), and high frequency activity (HF).

In some embodiments, the detecting is based, at least in part, on the one or more heart activity-related parameters.

In some embodiments, the control module is further configured to receive, as an additional input, data that is indicative of at least one additional parameter selected from the group consisting of: an activity status of the subject, a prandial status of the subject, and an emotional state of the subject.

In some embodiments, the control module is further configured to determine that the autonomous nervous system of the subject is in a sympathetic state or a parasympathetic state.

In some embodiments, when the autonomous nervous system of the subject is in a sympathetic state, the control module is configured to operate the neuro-stimulation unit to generate the electrical stimulation signal at a frequency of between 1-5 Hz.

In some embodiments, the system is housed within a wearable device configured to be worn by a subject, wherein the wearable device comprises a wristband configured to be worn around a wrist of the subject such that the at least two electrodes are each positioned along a lengthwise axis thereof, proximately, and substantially parallel to, a longitudinal axis of the median nerve.

In some embodiments, the arrhythmia-related condition is one or more of: atrial fibrillation (AF), premature atrial complex (PAC), premature ventricular complex (PVC), supraventricular tachycardia (SVT), atrial tachycardia (AT), atrial flutter, atrioventricular node re-entrant tachycardia (AVNRT), paroxysmal supraventricular tachycardia (PSVT), atrioventricular re-entrant tachycardia, Wolff-Parkinson-White syndrome, ventricular tachycardia (VT), torsades de pointes (TdP), long QT syndrome, heart block, and sick sinus syndrome.

In some embodiments, the control module is further configured to measure an electrical current associated with the electrical stimulation signal between the at least two electrodes, and to determine that a positioning of the at least two electrodes relative to the inner surface of the wrist of the subject is incorrect when the measured electrical current is lower than a predetermined baseline value.

In some embodiments, the predetermined baseline value is determined by measuring an electrical current associated with the electrical stimulation signal between the at least two electrodes, when the at least two electrodes are each positioned along a lengthwise axis thereof proximately, and substantially parallel, to a longitudinal axis of the median nerve.

In accordance with some applications of the present invention, a wearable device is configured to be placed on a wrist of the subject in a predefined configuration. At least some of the electrodes are wrist-facing electrodes, which are configured to be placed in contact with skin of the subject's wrist. The wrist-facing electrodes are typically disposed such that, when the wrist-wearable device is placed upon the subject's wrist in the predefined disposition, the wrist-facing electrodes are disposed in the vicinity of the subject's ulnar nerve and/or median nerve. For example, the device may be configured such that, when the device is placed on the subject's wrist with a screen facing upwards, the wrist-facing electrodes are disposed in the vicinity of the subject's ulnar nerve and/or median nerve.

It is noted that, although some of the sensing and/or neuromodulation techniques and the algorithms for use therewith are described herein with reference to a wearable device, the scope of the present invention includes practicing any one of the sensing and/or neuromodulation techniques and the algorithms for use therewith without the use of a wearable device. For example, such techniques and algorithms may be practiced using electrodes and/or sensors that are placed in contact with the subject's body in the absence of a wearable device, and/or using a wearable device that is worn on a portion of the subject's body other than the subject's wrist. In some embodiments, one or more of the subject's ulnar, median, radial, tibial, peroneal, subcostal, intravertebral nerves, and/or a different nerve is stimulated.

In some embodiments, the device is configured to provide a neuromodulation treatment by delivering an electrical signal from a first one of the wrist-facing electrodes to the other of the wrist-facing electrodes, such that the electrical signal passes through the subject's ulnar nerve and/or median nerve. (In some embodiments, the directionality of the signal alternates between the electrodes.) Typically, the device is configured to provide a neuromodulation treatment to a subject suffering from atrial fibrillation (AF) and/or arrhythmia conditions, such as occurrences of premature atrial contraction, premature ventricular contractions, supraventricular tachycardia, and/or atrial tachycardia. The signal typically has any one of a sinusoidal, triangular, rectangular, and/or saw tooth waveform. Typically, during a given treatment session, the signal is applied in an on-off duty cycle in which the signal is applied for between 5 and 30 seconds, before being switched off for a period of between 0 and 5 seconds.

The signal is typically driven at a frequency of 40-50 Hz (e.g., 40, 41, 42, 43, 44, 45, 46, 47, 48, 49, or 50 Hz). As evidenced by data presented herein, it has been found that such stimulation parameters typically reduce occurrences of AF and/or arrhythmia conditions (indicating an amelioration in cardiovascular distress and/or a reduction in cardiovascular deterioration). It has additionally been found by the inventors that stimulation of certain subjects at a frequency of between 1-5 Hz (e.g., 1, 2, 3, 4, or 5 Hz) reduces occurrences of AF and/or arrhythmia conditions (indicating an amelioration in cardiovascular distress and/or a reduction in cardiovascular deterioration). Therefore, In some embodiments, the signal is driven at a frequency of between 1-5 Hz (e.g., 1, 2, 3, 4, or 5 Hz). In some embodiments, the system selects which signal parameters to apply to the subject during a given treatment based upon one or more detected parameters and/or additional inputs.

There is therefore provided, in accordance with some applications of the present invention, apparatus including: a wearable device that is configured to be worn by a subject, the wearable device including one or more electrodes, and the wearable device being configured to detect a cardiac-related parameter of the subject; and at least one computer processor associated with the wearable device, the computer processor being configured to: receive the detected cardiac-related parameter of the subject, detect an increase in a rate of occurrences of premature atrial contractions and/or atrial tachycardia of the subject, based upon the detected cardiac-related parameter, at least partially in response thereto, drive the one or more electrodes of the wearable device to apply an electrical stimulation treatment to the subject having a frequency in a range selected from the group consisting of: between 40 Hz and 50 Hz, and between 1 Hz and 5 Hz.

In some applications, the wearable device includes a wristband, and at least one of the one or more electrodes is configured to be placed in a vicinity of a median nerve and/or an ulnar nerve of the subject, when the wristband is worn in a predefined disposition.

In some applications, the wearable device includes a PPG sensor and the wearable device is configured to detect the cardiac-related parameter of the subject by utilizing the PPG sensor.

In some applications, the wearable device includes an accelerometer and the wearable device is configured to detect the cardiac-related parameter of the subject by utilizing the accelerometer.

In some applications, the computer processor is additionally configured to receive an input that is indicative of at least one additional parameter selected from the group consisting of: an activity status of the subject, a prandial status of the subject, an emotional state of the subject, and a time of day, and the computer processor is configured to drive the one or more electrodes to apply the electrical stimulation treatment to the subject in response to the at least one additional parameter in combination with the detected increase in the rate of occurrences of premature atrial contractions and/or atrial tachycardia of the subject.

In some applications, the computer processor is further configured to determine that the subject is in a parasympathetic state, and the computer processor is configured to drive the one or more electrodes of the wearable device to apply the electrical stimulation treatment to the subject having the frequency of between 40 Hz and 50 Hz in response to detecting that the subject is in the parasympathetic state in combination with the detected increase in the rate of occurrences of premature atrial contractions and/or atrial tachycardia of the subject.

In some applications, the computer processor is further configured to determine that the subject is in a sympathetic state, and, in response to determining that the subject is in a sympathetic state, in combination with the detecting an increase in the rate of occurrences of premature atrial contractions and/or atrial tachycardia of the subject, the computer processor is configured to drive the one or more electrodes of the wearable device to apply an electrical stimulation treatment to the subject having a frequency of between 1 Hz and 5 Hz.

In some applications, the wearable device is configured to detect the cardiac-related parameter by recording an ECG of the subject, utilizing the electrodes.

In some applications, at least one of the electrodes is a dual-function electrode, which is configured to have a sensing mode in which the dual-function electrode is configured to record the subject's ECG, and to have a stimulation mode in which the computer processor is configured to apply an electrical stimulation treatment to the subject, via the dual-function electrode.

There is further provided, in accordance with some applications of the present invention, a method including: detecting a cardiac-related parameter of the subject, using a wearable device that includes one or more electrodes; and using at least one computer processor: receiving the detected cardiac-related parameter; detecting an increase in a rate of occurrences of premature atrial contractions and/or atrial tachycardia of the subject, based upon the detected cardiac-related parameter; and at least partially in response thereto, driving one or more electrodes of the wearable device to apply an electrical stimulation treatment to the subject having a frequency in a range selected from the group consisting of: between 40 Hz and 50 Hz, and between 1 Hz and 5 Hz.

There is further provided, in accordance with some applications of the present invention, apparatus including: a wearable device that is configured to be worn by a subject and to receive an input that is indicative of an episode having occurred to the subject; and at least one computer processor associated with the wearable device, the computer processor being configured to: predict an upcoming AF occurrence, at least partially in response to the input that is indicative of the episode having occurred, and generate an output in response thereto.

There is further provided, in accordance with some applications of the present invention, apparatus including: a wearable device that is configured to be worn by a subject, the wearable device including one or more electrodes and the wearable device being configured to detect a cardiac-related parameter of the subject; and at least one computer processor associated with the wearable device, the computer processor being configured to: receive the detected cardiac-related parameter; in response to the detected cardiac-related parameter, detect that the subject is undergoing AF; classify the AF as being a given category of AF; select an electrical stimulation treatment to apply to the subject, based upon the category of the AF; and drive the one or more electrodes of the wearable device to apply the selected electrical stimulation treatment to the subject.

In some applications, the computer processor is configured to detect episodes of premature atrial contraction and/or atrial tachycardia of the subject that occurred prior to the AF, to analyze the episodes of premature atrial contraction and/or atrial tachycardia of the subject, and classify the AF as being a given category of AF at least partially in response thereto.

In some applications, the wearable device includes a wristband, and at least one of the one or more electrodes is configured to be placed in a vicinity of a median nerve and/or an ulnar nerve of the subject, when the wristband is worn in a predefined disposition.

In some applications, the wearable device includes a PPG sensor and the wearable device is configured to detect the cardiac-related parameter of the subject by utilizing the PPG sensor.

In some applications, the wearable device includes an accelerometer and the wearable device is configured to detect the cardiac-related parameter of the subject by utilizing the accelerometer.

In some applications, the at least one computer processor is further configured to receive an input that is indicative of at least one additional parameter selected from the group consisting of: an activity status of the subject, a prandial status of the subject, an emotional state of the subject, and a time of day, and the computer processor is configured to classify the AF as being the given category of AF at least partially based upon the at least one additional parameter.

In some applications, the computer processor is configured to classify the AF as being a given category of AF by classifying the AF as being correlated with either a parasympathetic state of the subject or a sympathetic state of the subject.

In some applications, in response to classifying the AF as being correlated with a parasympathetic state of the subject, the computer processor is configured to select to apply an electrical stimulation treatment to the subject having the frequency of between 40 Hz and 50 Hz.

In some applications, in response to classifying the AF as being correlated with a sympathetic state of the subject, the computer processor is configured to select to apply an electrical stimulation treatment to the subject having the frequency of between 1 Hz and 5 Hz.

In some applications, the computer processor is configured to analyze a heart-rate-variability (HRV) of the subject within a given time period prior to an onset of the AF episode, and classify the AF as being correlated with either a parasympathetic state of the subject or a sympathetic state of the subject, at least partially based upon the analysis of the subject's HRV within the given time period prior to the onset of the AF episode.

In some applications, the computer processor is configured to analyze a heart rate of the subject within a given time period prior to an onset of the AF episode, and classify the AF as being correlated with either a parasympathetic state of the subject or a sympathetic state of the subject, at least partially based upon the analysis of the subject's heart rate within the given time period prior to the onset of the AF episode.

In some applications, the computer processor is configured to analyze a heart rate recovery of the subject within a given time period prior to an onset of the AF episode, and classify the AF as being correlated with either a parasympathetic state of the subject or a sympathetic state of the subject, at least partially based upon the analysis of the subject's heart rate recovery within the given time period prior to the onset of the AF episode.

In some applications, the at least one computer processor is further configured to receive an input that is indicative of at least one additional parameter selected from the group consisting of: an activity status of the subject, a prandial status of the subject, an emotional state of the subject, and a time of day, and the computer processor is configured to classify the AF as being correlated with either a parasympathetic state of the subject or a sympathetic state of the subject at least partially based upon the at least one additional parameter.

In some applications, the computer processor is configured to record an ECG of the subject using the electrodes, and classify the AF as being correlated with either a parasympathetic state of the subject or a sympathetic state of the subject, at least partially based upon the analysis of the ECG.

In some applications, the computer processor is configured to classify the AF as being correlated with either a parasympathetic state of the subject or a sympathetic state of the subject, at least partially based upon a shape of the ECG signal.

In some applications, the computer processor is configured to classify the AF as being correlated with either a parasympathetic state of the subject or a sympathetic state of the subject, at least partially based upon a regularity of beats in the ECG signal.

In some applications, the wearable device is configured to detect the cardiac-related parameter by recording an ECG of the subject, utilizing the electrodes.

In some applications, at least one of the electrodes is a dual-function electrode, which is configured to have a sensing mode, in which the electrode is configured to record the subject's ECG, and to have a stimulation mode, in which the computer processor is configured to apply an electrical stimulation treatment to the subject, via the electrode.

There is further provided, in accordance with some applications of the present invention, a method including: detecting a cardiac-related parameter of the subject, using a wearable device that includes one or more electrodes; and using at least one computer processor associated with the wearable device: receiving the detected cardiac-related parameter; in response to the detected cardiac-related parameter, detecting that the subject is undergoing AF; classifying the AF as being a given category of AF; selecting an electrical stimulation treatment to apply to the subject, based upon the category of the AF; and driving the one or more electrodes of the wearable device to apply the selected electrical stimulation treatment to the subject.

There is further provided, in accordance with some applications of the present invention, apparatus including: a wearable device that is configured to be worn by a subject, the device including: a photoplethysmography (PPG) sensor; and one or more electrodes; and at least one computer processor associated with the wearable device, the computer processor being configured to: receive data from the PPG sensor, and sense a heart rate of the subject in response thereto, based upon the sensed heart rate, detect that the subject has undergone and/or is undergoing a heart-rate related episode, at least partially in response to detecting the heart-rate-related episode, prompt the subject to record an electrocardiogram (ECG), at least partially based upon the ECG, determine that the subject is currently undergoing and/or is predicted to undergo an atrial-fibrillation episode, and treat the current and/or predicted atrial-fibrillation episode, by applying an electrical stimulation via the one or more electrodes.

In some applications, the wearable device includes a wristband, and at least one of the one or more electrodes is configured to be placed in a vicinity of a median nerve and/or an ulnar nerve of the subject, when the wristband is worn in a predefined configuration.

In some applications, the wearable device further includes an accelerometer and the computer processor is configured to prompt the subject to record the ECG at least partially based upon data acquired by the accelerometer.

In some applications, the wearable device further includes an accelerometer and the computer processor is configured to determine that the subject is currently undergoing and/or is predicted to undergo an atrial-fibrillation episode at least partially based upon data acquired by the accelerometer.

In some applications, the computer processor is configured to determine that the subject is predicted to undergo an atrial-fibrillation episode by detecting an increase in a rate of occurrences of premature atrial contractions and/or atrial tachycardia of the subject.

In some applications, the computer processor is further configured to receive an input that is indicative of at least one additional parameter selected from the group consisting of: an activity status of the subject, a prandial status of the subject, an emotional state of the subject, and a time of day, and the computer processor is configured to determine that the subject is currently undergoing and/or is predicted to undergo an atrial-fibrillation episode based upon the at least one additional parameter, in combination with the ECG.

In some applications, the computer processor is further configured to receive an input that is indicative of at least one additional parameter selected from the group consisting of: an activity status of the subject, a prandial status of the subject, an emotional state of the subject, and a time of day, and the computer processor is configured to classify the episode that the subject is currently undergoing and/or is predicted to undergo as being a given category of episode based upon the at least one additional parameter.

In some applications, the computer processor is further configured to determine that the subject is in a parasympathetic state, and the computer processor is configured to treat the current and/or predicted atrial-fibrillation episode by driving the one or more electrodes of the wearable device to apply an electrical stimulation treatment to the subject having the frequency of between 40 Hz and 50 Hz.

In some applications, the computer processor is further configured to determine that the subject is in a sympathetic state, and the computer processor is configured to treat the current and/or predicted atrial-fibrillation episode by driving the one or more electrodes of the wearable device to apply an electrical stimulation treatment to the subject having the frequency of between 1 Hz and 5 Hz.

In some applications, at least one of the electrodes is a dual-function electrode, which is configured to have a sensing mode, in which the dual-function electrode is configured to record the subject's ECG, and to have a stimulation mode, in which the computer processor is configured to apply the electrical stimulation treatment to the subject, via the dual-function electrode.

There is further provided, in accordance with some applications of the present invention, a method including: detecting a PPG signal of a subject, using a wearable device that includes one or more electrodes; and using at least one computer processor associated with the wearable device: receiving data from the PPG sensor, and sensing a heart rate of the subject in response thereto; based upon the sensed heart rate, detecting that the subject has undergone and/or is undergoing a heart-rate related episode; at least partially in response to detecting a heart-rate-related episode, prompting the subject to record an electrocardiogram (ECG); at least partially based upon the ECG, determining that the subject is currently undergoing and/or is predicted to undergo an atrial-fibrillation episode; and treating the current and/or predicted atrial-fibrillation episode, by applying an electrical stimulation via the one or more electrodes.

There is further provided, in accordance with some applications of the present invention, apparatus including: a wearable device that is configured to be worn by a subject, the wearable device including one or more electrodes, and the wearable device being configured to detect a cardiac-related parameter of the subject; and a computer processor associated with the wearable device, the computer processor being configured to: receive the detected cardiac-related parameter; identify an AF episode by analyzing the detected cardiac-related parameter, in response to identifying the AF episode, analyze the heart rate variability (HRV) of the subject within a given time period prior to an onset of the AF episode, select an electrical stimulation treatment to apply to the subject, at least partially based upon the analysis of the subject's HRV within the given time period prior to the onset of the AF episode, and drive the one or more electrodes of the wearable device to apply the selected electrical stimulation treatment to the subject.

In some applications, the wearable device includes a wristband, and at least one of the one or more electrodes is configured to be placed in a vicinity of a median nerve and/or an ulnar nerve of the subject, when the wristband is worn in a predefined configuration.

In some applications, the wearable device includes a PPG sensor and the wearable device is configured to detect the cardiac-related parameter of the subject by utilizing the PPG sensor.

In some applications, the wearable device includes an accelerometer and the wearable device is configured to detect the cardiac-related parameter of the subject by utilizing the accelerometer.

In some applications, the computer processor is additionally configured to receive an input that is indicative of at least one additional parameter selected from the group consisting of: an activity status of the subject, a prandial status of the subject, an emotional state of the subject, and a time of day, and the computer processor is configured to select the electrical stimulation treatment to apply to the subject based upon the at least one additional parameter in combination with the analysis of the subject's HRV within the given time period prior to the onset of the AF episode.

In some applications, at least partially based upon the analysis of the subject's HRV within the given time period prior to the onset of the AF episode, the computer processor is configured to classify the AF as being correlated with either a parasympathetic state of the subject or a sympathetic state of the subject.

In some applications, in response to classifying the AF as being correlated with a parasympathetic state of the subject, the computer processor is configured to select to apply an electrical stimulation treatment to the subject having the frequency of between 40 Hz and 50 Hz.

In some applications, in response to classifying the AF as being correlated with a sympathetic state of the subject, the computer processor is configured to select to apply an electrical stimulation treatment to the subject having the frequency of between 1 Hz and 5 Hz.

In some applications, the at least one computer processor is additionally configured to receive an input that is indicative of at least one additional parameter selected from the group consisting of: an activity status of the subject, a prandial status of the subject, an emotional state of the subject, and a time of day, and the computer processor is configured to classify the AF as being correlated with either a parasympathetic state of the subject or a sympathetic state of the subject based upon the at least one additional parameter in combination with the analysis of the subject's HRV within the given time period prior to the onset of the AF episode.

In some applications, the wearable device is configured to detect the cardiac-related parameter by recording an ECG of the subject, utilizing the electrodes.

In some applications, at least one of the electrodes is a dual-function electrode, which is configured to have a sensing mode, in which the electrode is configured to record the subject's ECG, and to have a stimulation mode, in which the computer processor is configured to apply an electrical stimulation treatment to the subject, via the electrode.

There is further provided, in accordance with some applications of the present invention, a method including: detecting a cardiac-related parameter of the subject, using a wearable device that includes one or more electrodes; and using at least one computer processor associated with the wearable device: receiving the detected cardiac-related parameter; identifying an AF episode by analyzing the detected cardiac-related parameter; in response to identifying the AF episode, analyzing the heart rate variability (HRV) of the subject within a given time period prior to an onset of the AF episode; selecting an electrical stimulation treatment to apply to the subject, at least partially based upon the analysis of the subject's HRV within the given time period prior to the onset of the AF episode; and driving the one or more electrodes of the wearable device to apply the selected electrical stimulation treatment to the subject.

There is further provided, in accordance with some applications of the present invention, apparatus including: a wearable device that is configured to be worn by a subject, the wearable device including one or more electrodes, and the wearable device being configured to detect a cardiac-related parameter of the subject; and a computer processor associated with the wearable device, the computer processor being configured to: receive the detected cardiac-related parameter; identify an AF episode by analyzing the detected cardiac-related parameter, in response to identifying the AF episode, analyze at least one physiological parameter of the subject within a given time period prior to an onset of the AF episode, the physiological parameter being selected from the group consisting of: heart rate, HRV, and heart rate recovery, select an electrical stimulation treatment to apply to the subject, at least partially based upon the analysis of the at least one physiological parameter within the given time period prior to the onset of the AF episode, and drive the one or more electrodes of the wearable device to apply the selected electrical stimulation treatment to the subject.

There is further provided, in accordance with some applications of the present invention, apparatus including: a wrist-wearable device configured to be placed on a wrist of the subject in a predefined configuration, the wrist-wearable device including one or more electrodes, and the electrodes being disposed such that, when the wrist-wearable device is placed upon the subject's wrist in the predefined disposition, the electrodes are disposed in a vicinity of a nerve selected from the group consisting of: an ulnar nerve, and a median nerve; and a computer processor configured to treat the atrial-fibrillation-related condition by delivering an electrical stimulation current into the selected nerve, via the electrodes, at a frequency of between 40 Hz and 50 Hz.

In some applications, the one or more electrodes include at least two wrist-facing electrodes that are disposed such that, when the wrist-wearable device is placed upon the subject's wrist in the predefined disposition, each of the two electrodes is disposed on a respective side of the selected nerve.

In some applications, each of the wrist-facing electrodes is shaped such as to have long edges and short edges, and a ratio between a length of the long edges to a length of the short edges is at least 3:2.

In some applications, the wrist-facing electrodes are disposed such that, when the wrist-wearable device is placed upon the subject's wrist in the predefined disposition, the long edges of the wrist-facing electrodes are substantially aligned with a direction of a length of the selected nerve.

There is further provided, in accordance with some applications of the present invention, a method including: identifying a subject as suffering from an atrial-fibrillation-related condition; in response thereto, placing a wrist-wearable device on a wrist of the subject in a predefined configuration, the wrist-wearable device including one or more electrodes, and the electrodes being disposed such that, when the wrist-wearable device is placed upon the subject's wrist in the predefined disposition, the electrodes are disposed in a vicinity of a nerve selected from the group consisting of: an ulnar nerve, and a median nerve; and treating the atrial-fibrillation-related condition by delivering an electrical stimulation current into the selected nerve, via the electrodes, at a frequency of between 40 Hz and 50 Hz.

There is further provided, in accordance with some applications of the present invention, apparatus including: a wearable device that is configured to be worn by a subject, the wearable device including a plurality of electrodes; and a computer processor associated with the wearable device, the computer processor being configured to: record an electrocardiogram (ECG) of the subject via the plurality of electrodes, reduce an incidence of AF of the subject by delivering an electrical stimulation current into a nerve of the subject; and condition the delivery of the electrical stimulation current into the subject's nerve upon an ECG having been recorded within a given time period prior to the delivery of the electrical stimulation current.

There is further provided, in accordance with some applications of the present invention, a method including: recording an electrocardiogram (ECG) of the subject, using a wearable device that includes one or more electrodes; and using at least one computer processor associated with the wearable device: receiving an electrocardiogram (ECG) of the subject; reducing an incidence of AF of the subject by delivering an electrical stimulation current into a nerve of the subject; and conditioning the delivery of the electrical stimulation current into the subject's nerve upon an ECG having been recorded within a given time period prior to the delivery of the electrical stimulation current.

There is further provided, in accordance with some applications of the present invention, apparatus including: a module including a plurality of electrodes; and a computer processor associated with the module, the computer processor being configured to: deliver electrical stimulation treatments into a subject's nerve via the electrodes, the electrical stimulation treatments being configured to reduce occurrences of AF and/or arrhythmia events, detect a physiological parameter of the subject before and after the electrical stimulation treatments are delivered, and determine outcomes of respective treatments in response thereto, detect events associated with respective treatments, the events being selected from the group consisting of: times of day of respective treatment, and prandial status of the subject during respective treatments, correlate outcomes of the respective treatments with events associated with the respective treatments, and optimize future treatments of the subject, based upon correlating the outcomes of respective treatments with events associated with the respective treatments.

There is further provided, in accordance with some applications of the present invention, a method including: delivering electrical stimulation treatments into a subject's nerve, the electrical stimulation treatments being configured to reduce occurrences of AF and/or arrhythmia events; detecting a physiological parameter of the subject before and after the electrical stimulation treatments are delivered, and determining outcomes of respective treatments in response thereto; detecting events associated with respective treatments, the events being selected from the group consisting of: times of day of respective treatment, and prandial status of the subject during the respective treatments; correlating outcomes of respective treatments with events associated with the respective treatments; and optimizing future treatments of the subject, based upon correlating the outcomes of respective treatments with events associated with the respective treatments.

There is further provided, in accordance with some applications of the present invention, apparatus including: one or more electrodes configured to be placed upon skin of a wrist of a subject in a vicinity of a nerve selected from the group consisting of: an ulnar nerve, and a median nerve; and a computer processor configured to: drive an electrical stimulation signal into the selected nerve via the electrodes, the electrical stimulation signal being configured to reduce occurrences of AF and/or arrhythmia events; in response to receiving an input indicating that the electrical stimulation signal does not cause any sensation in a vicinity of the electrodes, increase an intensity of the electrical stimulation signal; and in response to receiving an input indicating that the electrical stimulation signal causes involuntary movement of a portion of the subject's body in the vicinity of the electrodes, reduce an intensity of the electrical stimulation signal.

In some applications, the apparatus further includes a user interface, and the computer processor is configured to receive the input indicating that the electrical stimulation signal does not cause any sensation in a vicinity of the electrodes and/or the input indicating that the electrical stimulation signal causes involuntary movement of a portion of the subject's body in the vicinity of the electrodes from the subject via the user interface.

There is further provided, in accordance with some applications of the present invention, apparatus including: one or more electrodes configured to be placed upon skin of a wrist of a subject in a vicinity of a nerve selected from the group consisting of: an ulnar nerve, and a median nerve; and a computer processor configured to: drive an electrical stimulation signal into the selected nerve via the electrodes, the electrical stimulation signal being configured to reduce occurrences of AF and/or arrhythmia events; receive an input from the subject indicating a reaction of the subject's body in the vicinity of the electrodes to the electrical stimulation signal; and set an intensity of the electrical stimulation signal, such that the electrical stimulation signal causes a sensation in a vicinity of the electrodes, but does not cause involuntary movement of a portion of the subject's body in the vicinity of the electrodes.

In some applications, the apparatus further includes a user interface, and the computer processor is configured to receive the input from the subject via the user interface.

There is further provided, in accordance with some applications of the present invention, apparatus including: one or more electrodes configured to be placed upon skin of a wrist of a subject in a vicinity of a nerve selected from the group consisting of: an ulnar nerve, and a median nerve; and a computer processor configured to: drive an electrical stimulation signal into the selected nerve via the electrodes at a given voltage; detect a current of the electrical stimulation sign al, when the electrodes are in good contact with the skin of the subject's wrist; and in response to the current being below a threshold, generate an output indicating that at least one of the electrodes should be moved.

In some applications, in response to the current being below the threshold, the computer processor is configured to generate an output indicating that at least one of the electrodes should be moved closer to the selected nerve.

There is further provided, in accordance with some applications of the present invention, apparatus including: one or more electrodes configured to be placed upon skin of a wrist of a subject in a vicinity of a nerve selected from the group consisting of: an ulnar nerve, and a median nerve; and a computer processor configured to: drive an electrical stimulation signal into the selected nerve via the electrodes at a given current; detect a voltage of the electrical stimulation signal, when the electrodes are in good contact with the skin of the subject's wrist; and in response to the voltage being above a threshold, generate an output indicating that at least one of the electrodes should be moved.

In some applications, in response to the voltage being above the threshold, the computer processor is configured to generate an output indicating that at least one of the electrodes should be moved closer to the selected nerve.

There is further provided, in accordance with some applications of the present invention, apparatus including: one or more electrodes configured to detect an ECG signal of the subject; and a computer processor configured to: receive the detected ECG signal; analyze a multiple-heartbeat section of the detected ECG signal that corresponds to multiple heartbeats; analyze a QRS-complex section of the detected ECG signal that corresponds to an individual heartbeat; and identify and/or classify an AF and/or an arrhythmia event at least partially based upon a combination of analyzing the multiple-heartbeat section of the detected ECG signal and the QRS-complex section of the detected ECG signal.

In addition to the exemplary aspects and embodiments described above, further aspects and embodiments will become apparent by reference to the figures and by study of the following detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below.

FIGS. 1A-1B are schematic illustrations of an exemplary subject monitoring and treatment system that includes a wearable device and one or more external computing devices, in accordance with some applications of the present invention;

FIGS. 2A-2C are illustrations of alternative views of an exemplary wearable device, in accordance with some applications of the present invention;

FIGS. 2D-2E are schematic illustrations of module that is configured to apply a neuromodulation treatment to a subject and/or to monitor a subject, in accordance with some applications of the present invention;

FIG. 3A is a schematic cross-sectional illustration of a human wrist/forearm, showing the location of the median and ulnar nerves in relation to the inner skin surface of wrist/forearm, and a schematic exemplary module of the device of the present disclosure, comprising two stimulation electrodes, disposed about the inner skin surface of wrist/forearm, in accordance with some applications of the present invention;

FIG. 3B is a schematic illustration of a human wrist/forearm having a device of the present disclosure wound and closed around it, in accordance with some applications of the present invention;

FIGS. 3C-3D show additional alternatives for arranging the stimulation electrodes of the present disclosure relative to the median nerve in accordance with some applications of the present invention;

FIG. 4 is a schematic diagram of the functional steps in a method for monitoring and treating a subject, in accordance with some applications of the present invention, using, e.g., the exemplary system described with reference to FIGS. 1A-1B;

FIGS. 5A-5D are graphs showing experimental results of stimulation treatments that were performed on atrial fibrillation subjects, in accordance with some applications of the present invention; and

FIG. 6 is a graph showing experimental results of stimulation treatments that were performed on atrial fibrillation subjects, in accordance with some applications of the present invention.

### DETAILED DESCRIPTION

Disclosed herein are systems and methods for administering a neuromodulation treatment to a subject, through the delivery of electrical energy in the form of an electrical stimulation signal generated according to specified signal parameters, to reduce and/or mitigate occurrences of atrial fibrillation (AF), additional arrhythmia conditions, and/or additional cardiovascular conditions.

As used herein, ‘treatment’ may refer to any altering, modification, effecting, modulating, and/or handling action for one or more physiological conditions, states, properties, characters, functions, and/or activities.

As used herein, ‘electrical neuromodulation,’ ‘neuromodulation,’ and/or ‘neuro-stimulation’ may be used interchangeably to refer to electrical, electromagnetic, or electromechanical neuro-modulating of neural structures or nerves, e.g., low-level electrical nerve stimulation. In some embodiments, the neuromodulation is performed in a non-invasive or non-intrusive way, i.e., via transcutaneous or transdermal delivery of electrical energy to the nerve structure.

In some embodiments, the present disclosure provides for detecting a current and/or an oncoming AF or another arrhythmia-related condition in a subject, e.g., atrial tachycardia (AT), an acute increase in atrial premature complexes/premature atrial complexes (APC or PAC) burden or premature ventricular complexes/ventricular premature complexes (VPC or PVC). In some embodiments, an oncoming AF or another arrhythmia-related condition may be detected based, at least in part, on a detected specified precursor, e.g., an increase in PACs in a subject, e.g., premature heartbeats which originate in the atria, or the two upper chambers of the heart.

As used herein, an ‘arrhythmia-related condition’ or ‘arrhythmia-related event’ may refer broadly to any one or more of AF, premature atrial complexes (APC/PAC), acute premature atrial complex, premature ventricular complexes (VPC/PVC), supraventricular tachycardia (SVT), atrial tachycardia (AT), atrial flutter, atrioventricular node re-entrant tachycardia (AVNRT), Paroxysmal supraventricular tachycardia (PSVT), atrioventricular re-entrant tachycardia, Wolff-Parkinson-White syndrome, ventricular tachycardia (VT), torsades de pointes (TdP), long QT syndrome, heart block, and/or sick sinus syndrome.

In some embodiments, detection of the onset of an arrhythmia-related event may be based, at least in part, on continuous, periodic, or intermittent monitoring and analysis of cardiac activity in the subject, e.g., monitoring of heartrate and cardiac cycle in the subject. As used herein, ‘cardiac activity,’ ‘heart function’, ‘heart activity’ may be used interchangeably to denote various to parameters used to evaluate, estimate, and/or quantify heart-rate and heart-rate variability. In some embodiments, cardiac activity may be monitored using any suitable one or more cardiac activity sensors e.g., an electrocardiogram (ECG), which may be a potable ECG sensor, e.g., a Holter monitor, a photoplethysmogram (PPG) sensor, or otherwise.

In some embodiments, upon such detection of a current and/or an oncoming arrhythmia-related condition, the present disclosure then provides administering a specified course of treatment of neuromodulation of peripheral nerves of the subject, e.g., one or more of the median, ulnar, and/or radial nerves in the subject. In some embodiments, the present disclosure provides for administering the course of neuromodulation treatment to the median nerve of the subject.

By way of background, neuromodulation of the median and/or ulnar nerves plays a key role in both sympathetic and parasympathetic activity of the autonomic nervous system. Thus, the stimulation of the median and/or ulnar nerves may enhance parasympathetic activity of the vagus nerve and depress sympathetic activity of the cardiac nerve, and thereby reduce heart rate, regulate heart rhythm, and correct and prevent cardiac arrhythmias. Neuromodulation of the median and ulnar nerves may also induce a neurohormonal response: secretion of endorphins, decrease of stress related hormones, and thereby reduce stress and improve sleep.

The term ‘stimulation signal’, with reference to electrical neuro-stimulation, refers to a voltage/current signal applied between a pair of electrodes (attached to a target body part, such as to electrically neuro-stimulate at least one nerve therein the target body part).

In some embodiments, the stimulation signal of the present disclosure is applied to the median and/or ulnar nerves in the forearm of a subject. In some embodiments, the stimulation signal may be applied via one or more pairs of electrodes configured to attach to a specified bodily site of the subject and to deliver the signal to the median and/or ulnar nerves.

In some embodiments, the stimulation of peripheral nerves, e.g., the median nerve, is performed in a noninvasive manner, by transcutaneous or transdermal delivery of electrical energy in the form of an electrical stimulation signal generated according to specified signal parameters.

In some embodiments, the particulars of the course of treatment and the specified signal parameters may be determined based on analyzing one or more cardiac activity-related parameters in the subject, for example one or more of heart rate variability (HRV), heart rate recovery, heart rate reserve, premature atrial contractions, ventricular premature contractions, as well as HRV-related parameters such as RR intervals, average interval between normal heart beats (AVNN), standard deviation of NN intervals (SDNN), root mean square of successive differences between normal heartbeats (rMSSD), pNN50, low frequency activity (LF), and high frequency activity (HF).

In some embodiments, the particulars of the course of treatment and the specified signal parameters may be further determined based on subject state-related parameters, such as an activity status of the subject, a prandial status of the subject, and an emotional state of the subject, wherein at least some of these data may be derived from subject monitoring or otherwise may be self-reported by the subject.

In some embodiments, the present disclosure may further use the cardiac activity-related parameters and/or the data and/or the subject-related parameters to detect an activation state of specific components of the autonomous nervous system of the subject, e.g., the sympathetic nervous system (SNS) and/or the parasympathetic nervous system (PNS). In some embodiments, the present disclosure may be further configured to correlate the arrythmia-related condition with either a sympathetic or a parasympathetic state in the subject, wherein the particulars of the course of treatment and the specified signal parameters may be further determined based on this correlation.

In some embodiments, the particulars of the course of treatment may include, but are not limited to, timing of treatment session(s), number and overall duration of treatment session(s), and/or number and duration of on/off treatment session(s) cycles In some embodiments, specified signal parameters may be determined are determined based, at least in part, on

In some embodiments, the specified parameters of the stimulation signal, or ‘stimulation signal parameters,’ may include broadly any parameters characterizing an electrical stimulation signal applied between pairs of electrodes, including, but not limited to, overall signal duration, signal waveform (e.g., sinusoidal, triangular, rectangular, and/or saw tooth waveform), signal amplitude (e.g., maximum, minimum, mean, and/or average amplitude), signal intensity (e.g., maximum, minimum, mean, and/or average intensity), signal frequency, signal pulse parameters when the stimulation signal comprises a series of pulses (e.g., pulse duration and/or inter-pulse time intervals), and/or parameters characterizing the synchronization of the stimulation signal with one or more other signals and/or reference activities (e.g., cardiac activity of the subject).

Accordingly, in some embodiments, upon detection of a current or oncoming arrythmia-related condition in the subject, the present disclosure may be configured to determine the particulars of the course of treatment and/or the specified signal parameters, based at least in part, on the detected cardiac activity-related parameters and/or subject state-related parameters. In some embodiments, the present disclosure is then configured to apply the course of treatment to the subject, by delivering the stimulation signal according to the determined course of treatment, wherein the stimulation signal is delivered non-invasively to at least the median nerve of the subject, and wherein the simulation signal has a frequency of between 40-50 Hz (e.g., 40, 41, 42, 43, 44, 45, 46, 47, 48, 49, or 50 Hz).

In some embodiments, when the arrythmia-related condition is correlated with a parasympathetic state in the subject, the stimulation signal is configured to be delivered at a frequency of between 40-50 Hz (e.g., 40, 41, 42, 43, 44, 45, 46, 47, 48, 49, or 50 Hz). As evidenced by data presented herein, it has been found that such stimulation signal frequency, in conjunction with additional stimulation signal parameters, typically reduce occurrences of an arrhythmia-related condition in a subject in a parasympathetic state, thus indicating an amelioration in cardiovascular distress and/or a reduction in cardiovascular deterioration.

In some embodiments, when the arrythmia-related condition is correlated with a sympathetic state in the subject, the stimulation signal is typically delivered at a frequency of between 1-5 Hz (e.g., 1, 2, 3, 4, or 5 Hz). As evidenced by data presented herein, it has been found that such stimulation signal frequency, in conjunction with additional stimulation signal parameters, reduces occurrences of an arrhythmia-related condition in a subject in a sympathetic state, thus indicating an amelioration in cardiovascular distress and/or a reduction in cardiovascular deterioration.

By way of background, the sympathetic nervous system (SNS) is generally activated in response to mental or physical stress, and may be associated with increases in blood pressure, increases in blood flow to the muscles and lungs, decreases in blood flow to the digestive and reproductive systems, and the release of stress hormones and glucose to provide quick energy. In contrast, the parasympathetic nervous system (PNS) is typically activated in a state of recovery, such as during rest or sleep, after a meal, and is associated with slower heart rate and respiration, reduction in blood pressure, increase in intestinal activity and blood flow to the digestive tract, and a reduction in stress hormones and the release of neurotransmitters like acetylcholine, which regulates muscle contractions.

Accordingly, in some embodiments, a detected activation of the SNS in the subject may lead to a determination that the arrhythmia-related condition is correlated with a ‘sympathetic state’ in the subject. A detected activation of the PNS in the subject may lead to a determination that the arrhythmia-related condition is correlated with a ‘parasympathetic state’ in the subject.

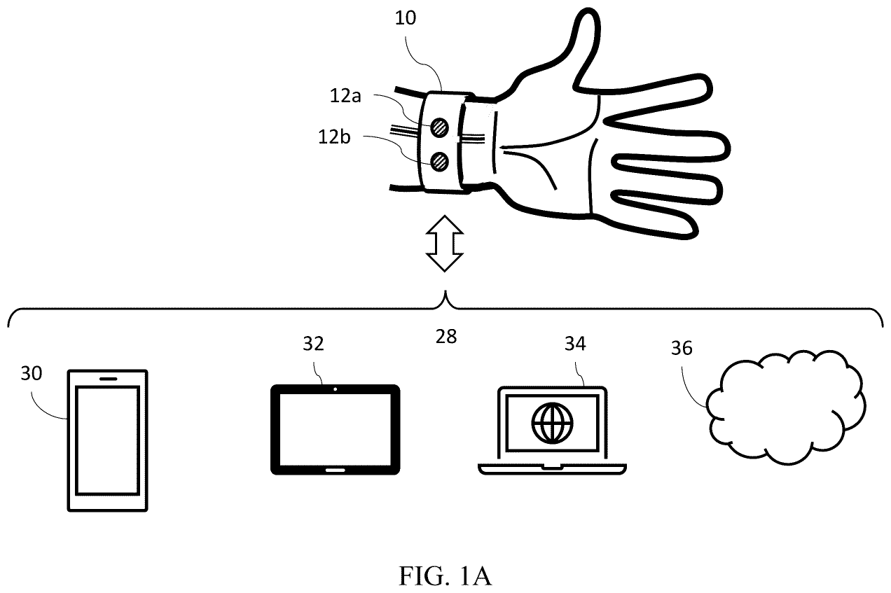

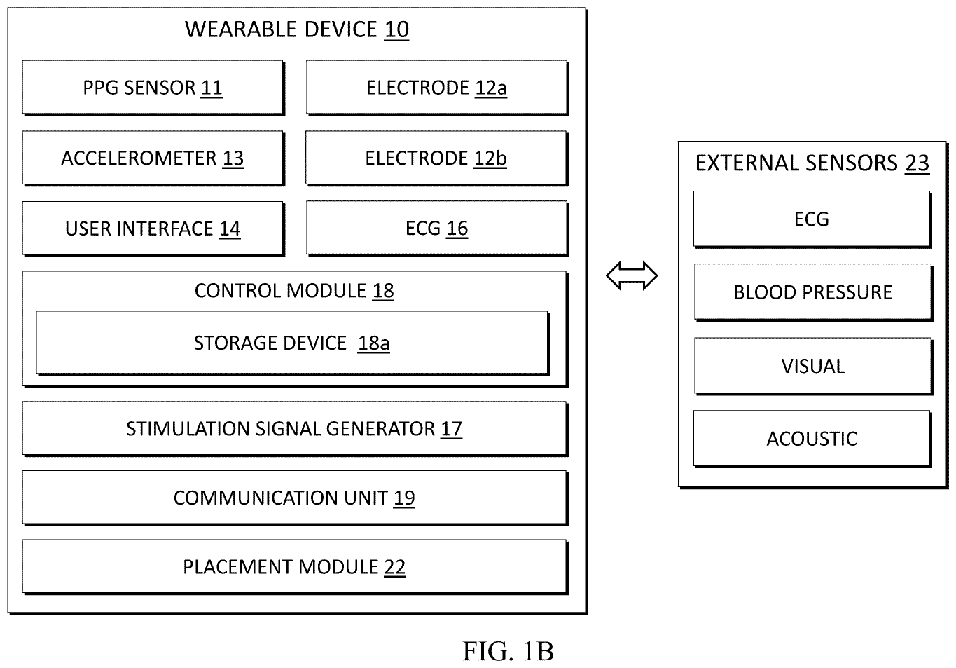

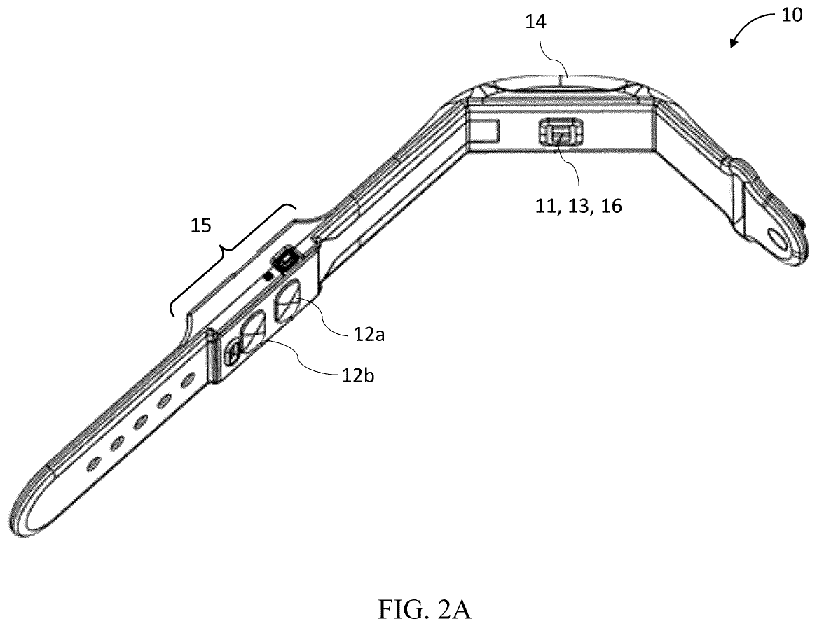

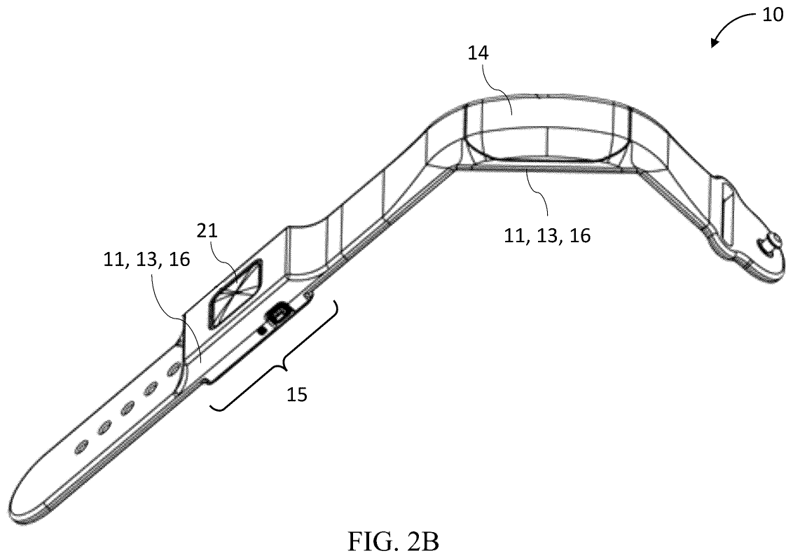

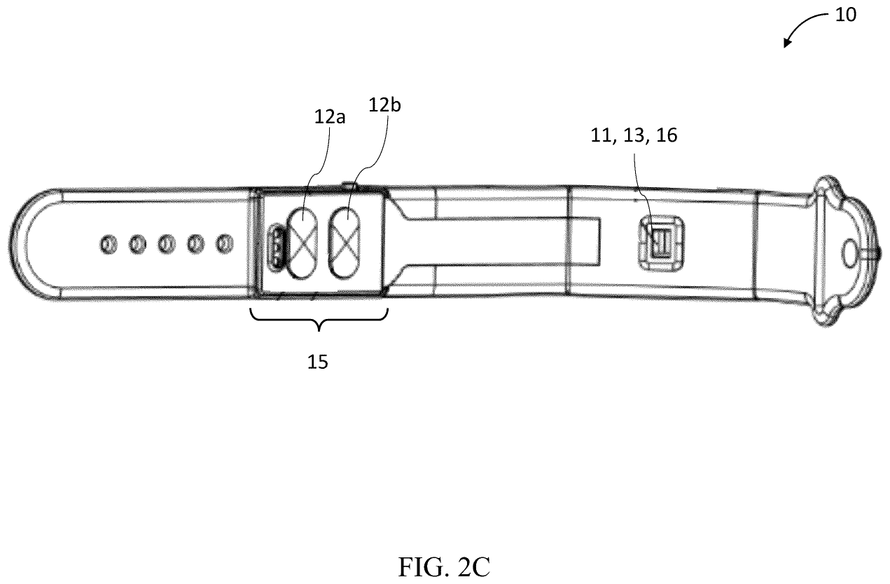

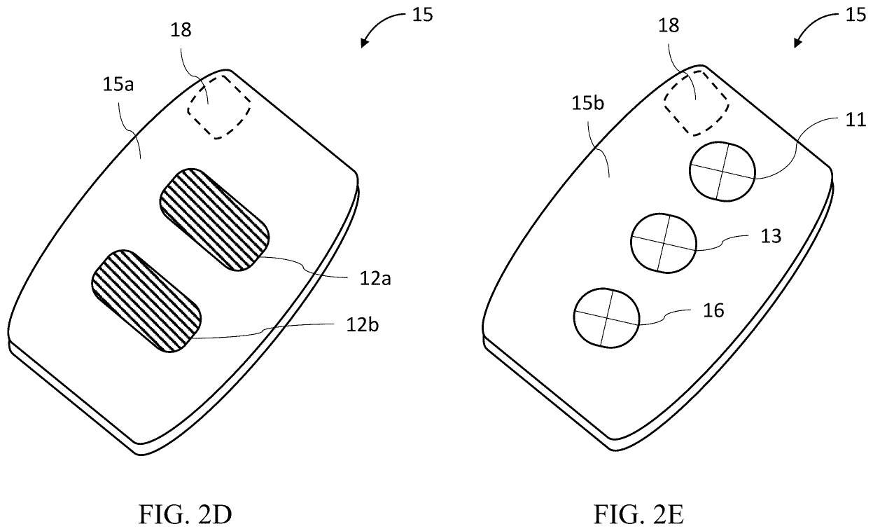

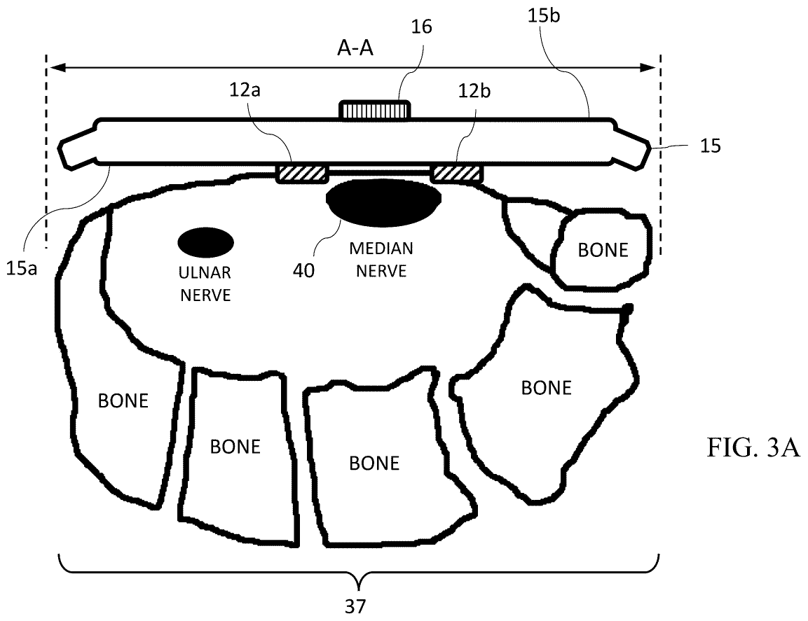

FIGS. 1A-1B are schematic illustrations of an exemplary subject monitoring and treatment system that includes a wearable device 10 and one or more external computing devices 28, in accordance with some applications of the present invention. Reference is also made to FIGS. 2A-2C which are illustrations of alternative views of an exemplary wearable device 10, in accordance with some applications of the present invention.

In some embodiments, wearable device 10 is configured to be worn on a subject's wrist, for example, with the wearable device configured as a wristband (as shown), a bracelet, a band, a cuff, a ring, a belt, a collar, or a chain. The device or a portion thereof (e.g., module 15 as shall be detailed below) may be embedded within a clothing article (e.g., sleeve of a shirt), or within a glove or a sock, or otherwise removably attached to the body of the subject at a desired body site. In some embodiments, the device or a portion thereof (e.g., module 15) may be independently held using a removable patch, sticker, or adhesive bandage. As used herein, an object may be referred to as being ‘independently held’ against a body part when—following the placing thereof against, or the attaching thereof to, the body part—the object is kept in place without any action on the part of the user. Alternatively or additionally, the wearable device is configured to be worn on a different portion of the subject's body.

In some embodiments, device 10 is configured to perform subject monitoring and subject treatment (i.e., stimulation) functions by switching/alternating between a monitoring mode and a treatment mode. For example, following a (preset) period of stimulation, the device may automatically switch back to the monitoring mode to obtain updated values of the monitored data and outcomes of the stimulation. The stimulation parameters are accordingly adjusted (i.e. taking into account the updated values). The device may then switch back to the stimulation mode and resume applying stimulation, and so on.

Typically, the wearable device includes one or more electrodes 12a, 12b, which are configured to provide a neuromodulation treatment to a subject. In some embodiments, at least one of the electrodes may be configured to also perform one or more sensing functionalities. For example, at least one electrode may be configured to record the subject's electrocardiogram (ECG). In some embodiments, at least one of the electrodes is a dual-function electrode, which is configured to perform neuromodulation functionalities in a first mode, and to perform sensing or monitoring functionalities in a second mode. Typically, the wearable device includes one or more additional sensors, such as an optical sensor (e.g., a photoplethysmogram (PPG) sensor 11), an accelerometer 13, and/or an ECG sensor 16.

Typically, the wearable device includes a user interface 14 which may include a display screen, which is optionally a touchscreen. User interface 14 is used to provide outputs to a user, and/or receive inputs from the user. In some embodiments, the wearable device includes a control module 18 (comprising, e.g., one or more hardware processors). In some embodiments, at least the electrodes 12a, 12b, as well as one or more of PPG sensor 11, accelerometer 13, the ECG sensor 16, and control module 18 are housed in detachable module 15 of device 10, as shall be further described below.

Control module 18 may include one or more hardware processor(s), a random-access memory (RAM), and one or more non-transitory computer-readable storage device(s), such as storage device 18a. Components of control module 18 may be co-located or distributed, or control module 18 may be configured to run as one or more cloud computing ‘instances,’ ‘containers,’ ‘virtual machines,’ or other types of encapsulated software applications, as known in the art.

Storage device 18a may have stored thereon program instructions and/or components configured to operate control module 18. The program instructions may include one or more software modules, which may include an operating system having various software components and/or drivers for controlling and managing general system tasks (e.g., memory management, storage device control, power management, etc.), and facilitating communication between various hardware and software components. Control module 18 may operate by loading instructions of the various software modules into its RAM as they are being executed by the hardware processor(s) comprising control module 18.

Control module 18 as described herein is only an exemplary embodiment of the present invention, and in practice may be implemented in hardware only, software only, or a combination of both hardware and software. Control module 18 may have more or fewer components and modules than shown, may combine two or more of the components, or may have a different configuration or arrangement of the components. Control module 18 may include any additional component enabling it to function as an operable computer system, such as a motherboard, data busses, power supply, a network interface card, a display, an input device (e.g., user interface 14), etc. Moreover, components of control module 18 may be co-located or distributed, or the system may be configured to run as one or more cloud computing ‘instances,’ ‘containers,’ ‘virtual machines,’ or other types of encapsulated software applications, as known in the art.

Typically, the wearable device or any module thereof (e.g., module 15) is configured to communicate with one or more external computing devices 28 (shown in FIGS. 1A-1B), which may include a computer processor of a local computing device such as a smartphone 30, a tablet device 32, and/or a personal computer 34, and/or a remote computing device or a remote server, e.g., a cloud-based remote server 36. For example, external computing devices may receive data from control module 18 of the wearable device, and may then process the data, for example, using techniques as described herein. Alternatively or additionally, the external computing device may send data and/or instructions to control module 18 of the wearable device. The present specification and claims include references to certain functionalities being performed by a computing device. Typically, such functionalities are performed by control module 18, external computing devices 28, and/or a combination thereof.

In some embodiments, device 10 may include a stimulation signal generator 17, including an electric power source (not shown). stimulation signal generator 17 is configured to generate an electric signal (e.g. a voltage signal or an electric current signal) between stimulation electrodes 12a, 12b, based on signal parameters determined by device 10. The stimulation parameters may specify the time-dependence of the generated electric signals. In particular, in some embodiments, stimulation signal generator 17 is configured to automatically adjust the voltage and frequency applied between stimulation electrodes 12a, 12b, such as to achieve a desired signal parameters. Stimulation signal generator 17 may include, for example, an AC current source or an electric signal generator, such that an intensity, a frequency, and a waveform of the voltage/current generated by stimulation signal generator 17 (including parameters characterizing a voltage/current including a continuous voltage/current pulse, intermittent voltage/current pulses, and burst modes) may be controllably varied in accordance with the stimulation parameters, thereby effecting neuromodulation of at least one nerve in a target body part as described herein.

Communication unit 19 is configured to send information to, and/or receive information from, an external agent, such as a smartphone, smartwatch, smart-band, a tablet, a personal computer, and/or a remote server. The sending and the receiving of information (e.g. monitored heart parameters, determined stimulation parameters) may be implemented wirelessly and/or by wire. Communication unit 19 may be configured for long-distance communication (e.g. to a user's cloud storage, a user's personal computer, a computer system of the user's healthcare provider, and/or a personal computer of the user's physician/caregiver), by means of cellular networks, and/or Wi-Fi. Alternatively or additionally, communication unit 19 may be configured for short-range communication (e.g. to the user's smartphone or tablet), for example, by means of Bluetooth, NFC (near field communication) technology, and/or Wi-Fi. In particular, in some embodiments, communication unit 19 may be used to relay information from device 10 to a user's smartphone/smartwatch and to relay commands from the user's smartphone/smartwatch to device 10, thereby facilitating controlling and operating by means of the smartphone/smartwatch.

In some embodiments, device 10 is configured to send information to, and to receive information from, control module 18 and/or an external agent (or an external device), such as a user's smartphone, smartwatch, home hub, by wireless communication or by wired communication (e.g. by a USB cable). Measurement data may be sent to control module 18 and/or external computing device 28, such as smartphone 30, tablet 32, computer 34, or a remote cloud server 36, or the like, wherein the measurement data is processed. In some embodiments, the determination of stimulation parameters may also be performed by control module 18 and/or external agent using the obtained values of the monitored data. The obtained values of the monitored data and the stimulation parameters may be stored in a memory on the external agent for future reference. Stimulation parameters may also be transmitted from the external agent directly to device 10 independently of the acquired data, e.g. preset parameters of a certain stimulation regiment/protocol to achieve a desired physiological effect.

In some embodiments, the wearable device and/or the external computing devices are configured to receive data from one or more external sensors 23 (shown in FIG. 2B) and/or one or more additional sensors that are built into the wearable device, such as an acoustic sensor, an optical sensor, a pressure sensor (e.g., configured to measure blood pressure), and/or a visual sensor.

Typically, wearable device 10 is configured to be placed on a wrist of the subject in a predefined configuration. Referring to FIG. 2C, which is a schematic illustration of a view of a wrist-facing side of the wearable device (i.e., a side of the wearable device that is configured to face the subject's wrist), it is noted that electrodes 12a, 12b are wrist-facing electrodes, which are configured to be placed in contact with skin of the subject's wrist. Wrist-facing electrodes are typically disposed such that, when the wrist-wearable device is placed upon the subject's wrist in the predefined disposition, the wrist-facing electrodes are disposed in the vicinity of the subject's median or another upper extremity peripheral nerve. For example, in the configuration of the device as shown in FIGS. 1A-1B, the device is configured such that, when the device is placed on the subject's wrist with a screen facing upwards, wrist-facing electrodes 12a, 12b are disposed in the vicinity of the subject's median or another upper extremity peripheral nerve.

In some embodiments, wearable device 10 is configured to be placed or held against or placed on a target body part of a subject such that stimulation electrodes 12a, 12b are both in skin or dermal contact with the target body part. As used herein, ‘skin contact,’ or ‘dermal contact’ between a conducting object and a body part may refer to direct contact between skin on the body part and the conducting object, as well as to indirect contact between the skin on the body part and the conducting object (for example, via a gel layer, saline solution, or the like, in between the object and the body part, or a medium carrying or soaked with such liquids), which allows for electrically associating the conducting object with the body part.

It is noted that, although some of the sensing and/or neuromodulation techniques and the algorithms for use therewith are described herein with reference to wearable device 10, the scope of the present invention includes practicing any one of the sensing and/or neuromodulation techniques and the algorithms for use therewith without the use of wearable device 10. For example, such techniques and algorithms may be practiced using electrodes and/or sensors that are placed in contact with the subject's body in the absence of a wearable device, and/or using a wearable device that is worn on a portion of the subject's body other than the subject's wrist. In some embodiments, one or more of the subject's median, ulnar radial, tibial, peroneal, subcostal, intravertebral nerves, and/or a different nerve is stimulated.

Typically, wearable device 10 includes a pair of wrist-facing electrodes 12a, 12b. In some embodiments, device 10 is configured to provide a neuromodulation treatment by delivering an electrical signal from a first one of the wrist-facing electrodes 12a, 12b to the other one of the wrist-facing electrodes 12a, 12b, such that the electrical signal is delivered to the subject's median nerve and/or another peripheral nerve such as the median nerve, via skin contact. As described hereinbelow, In some embodiments, the signal is biphasic, in which case the direction in which the signal is driven between the electrodes alternates. Referring again to FIG. 2C, In some embodiments, wrist-facing electrodes 12a, 12b are shaped such that the electrodes have an oblong rectangular, elliptical, semi-circular, or racetrack shaped outline that defines long and short axes of the electrode. A ratio of the length of the lengthwise axis to that of the transverse axis is typically at least 3:2 (e.g., at least 2:1). Typically, the wrist-facing electrodes 12a, 12b are oriented such that, when the wrist-wearable device is placed upon the subject's wrist in the predefined disposition, the lengthwise axis of each electrode 12a, 12b is disposed substantially parallel to a longitudinal axis of the subject's median or another upper extremity peripheral nerve. In this manner, the wearable device is configured such as to drive the electrical signal from the first wrist-facing electrode to the second-wrist facing electrode via the median or another upper extremity peripheral nerve, but substantially without leakage of the signal to other tissue and/or nerves in the vicinity of the electrodes.

Typically, wearable device 10 is configured to provide a neuromodulation treatment to a subject suffering from an arrhythmia-related condition, such as occurrences of AF, acute PACs, PVCs, supraventricular tachycardia, and/or atrial tachycardia. Typically, the treatment is provided by device 10 delivering an electrical signal from a first one of the wrist-facing electrodes 12a, 12b to the other of the wrist-facing electrodes, such that the electrical signal passes through the subject's median or another upper extremity peripheral nerve, as described hereinabove. As described hereinbelow, In some embodiments, the signal is biphasic, in which case the direction in which the signal is driven between the electrodes alternates. The signal typically has any one of a sinusoidal, triangular, rectangular, and/or saw tooth waveform. Typically, during a given treatment session, the signal is applied in an on-off duty cycle in which the signal is applied for between 5 and 30 seconds, before being switched off for a period of between 0 and 5 seconds.

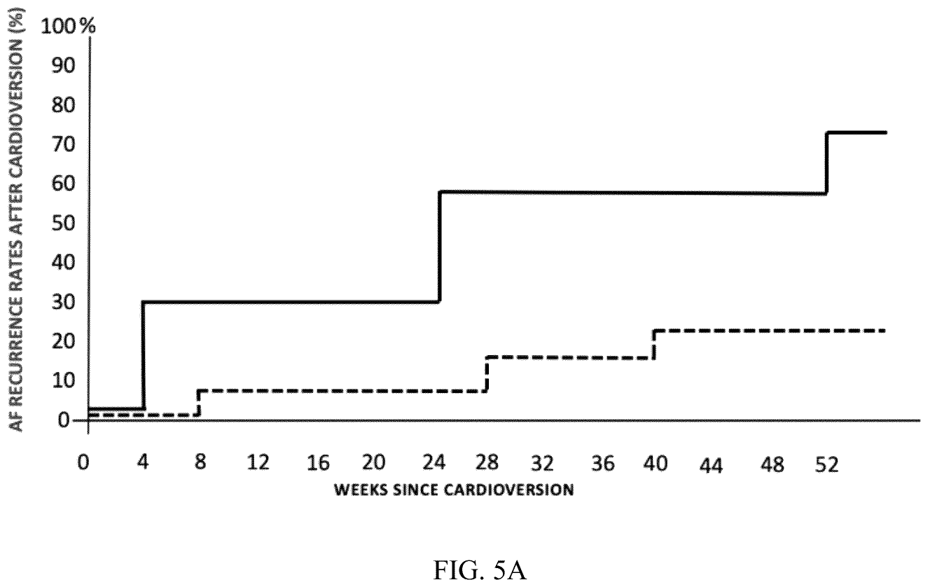

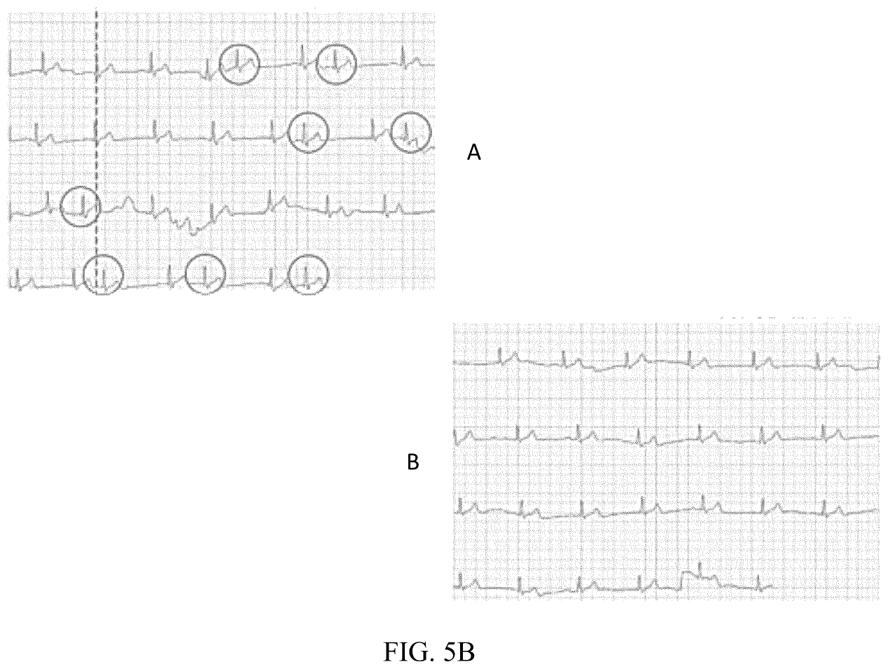

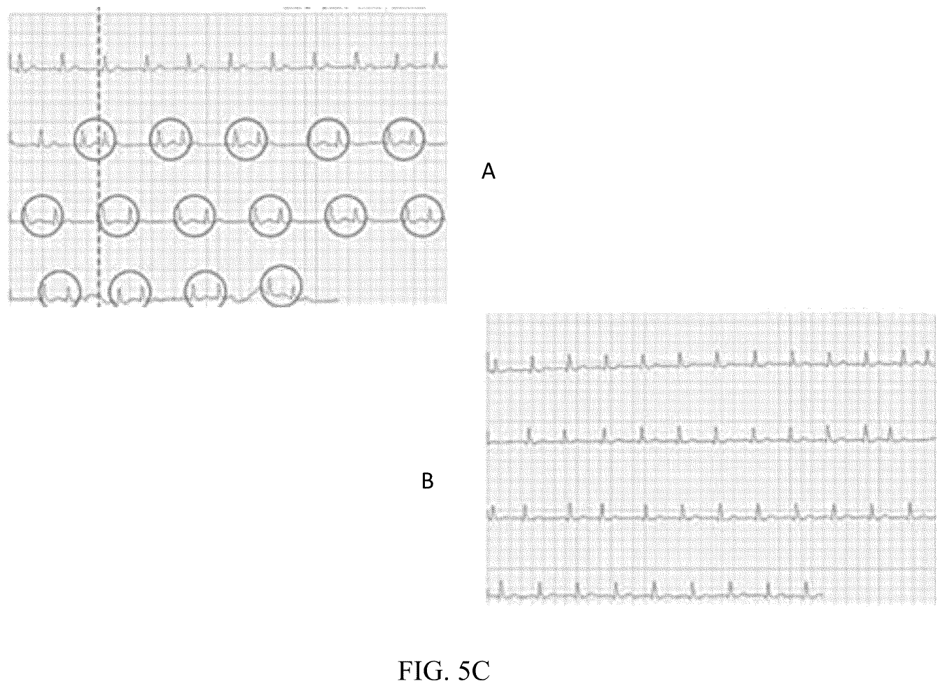

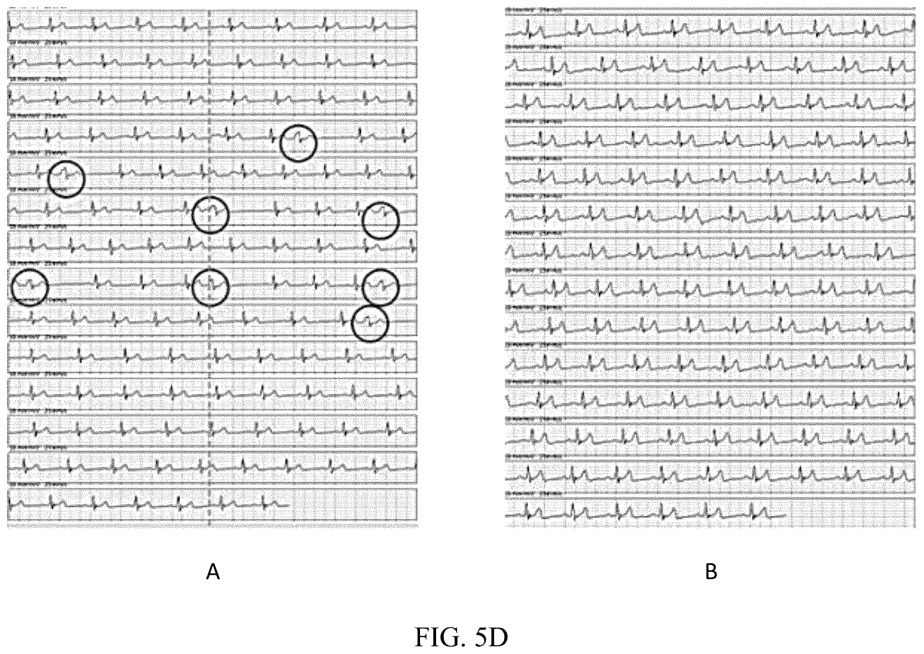

The signal is typically driven at a frequency of between 40-50 Hz (e.g., 40, 41, 42, 43, 44, 45, 46, 47, 48, 49, or 50 Hz or any frequency in between). As evidenced by data presented herein (and as described hereinbelow with reference to FIGS. 5A-5D), it has been found that such stimulation parameters typically reduce occurrences of AF and/or arrhythmia conditions (indicating an amelioration in cardiovascular distress and/or a reduction in cardiovascular deterioration). It has additionally been found by the inventors that stimulation of certain subjects at a frequency of between 1-5 Hz (e.g., 1, 2, 3, 4, or 5 Hz or any fractional frequency in between) reduces occurrences of AF and/or arrhythmia conditions (indicating an amelioration in cardiovascular distress and/or a reduction in cardiovascular deterioration). Therefore, In some embodiments, the signal is driven at a frequency of between 1-5 Hz (e.g., 1, 2, 3, 4, or 5 Hz). In some embodiments, the system selects which signal parameters to apply to the subject during a given treatment based upon one or more detected parameters and/or additional inputs, as described in further detail hereinbelow.

As further detailed herein, in many subjects, stimulation at 40-50 Hz was found to reduce occurrences of AF and/or arrhythmia conditions. Also as noted, in some subjects stimulation at 1-5 Hz was found to reduce occurrences of AF and/or arrhythmia conditions. It has been shown that stimulation at 40-50 Hz reduces occurrences of AF and/or arrhythmia conditions that is correlated with a parasympathetic state or vagal tone (which may be the case with subject's suffering from idiopathic AF and/or arrhythmia conditions), and that the stimulation at such frequencies restores the autonomic balance of such subjects. It is further has been shown that stimulation at 1-5 Hz reduces occurrences of AF and/or arrhythmia conditions that is correlated with excess sympathetic tone (which may be the case with subject's suffering from non-idiopathic AF and/or arrhythmia conditions, such as subjects with scarring on their heart, and/or CHF subjects or with permanent AF patients) and that stimulation at such frequencies restores the autonomic balance of such subjects.

In view of the above, In some embodiments, in response to detecting that the subject's autonomic system is currently unbalanced, stimulation is delivered such as to restore the subject's autonomic balance, irrespective of whether the subject is undergoing an AF and/or arrythmia episode, in order to reduce risks of the subject undergoing other clinical episodes. For example, if a subject's parasympathetic tone is too high and/or their sympathetic tone is too low, this can give rise to bradycardic arrythmias, and/or hypotension. In some embodiments, the system detects that the subject's parasympathetic tone is too high and/or their sympathetic tone is too low, for example, by detecting that their HRV is high, and/or based on the subject's heart rate or heart rate recovery. In response to detecting that this is the case, the subject is stimulated with a signal having a frequency of 40-50 Hz (e.g., 40, 41, 42, 43, 44, 45, 46, 47, 48, 49, or 50 Hz or any fractional frequency in between), in order to restore autonomic balance, by decreasing parasympathetic tone, and/or increasing sympathetic tone. Conversely, if a subject's sympathetic tone is too high and/or their parasympathetic tone is too low, this can give rise to tachycardiac arrhythmias, hypertension, heart failure exacerbation, sleep apnea, and/or impaired recovery from myocardial infarction. In some embodiments, the system detects that the subject's sympathetic tone is too high and/or that their parasympathetic tone is too low, for example, by detecting that their HRV is low, and/or based on the subject's heart rate or heart rate recovery. In response to detecting that this is the case, the subject is stimulated with a signal having a frequency of between 1-5 Hz (e.g., 1, 2, 3, 4, or 5 Hz or any fractional frequency in between), in order to restore autonomic balance, by decreasing sympathetic tone, and/or increasing parasympathetic tone.

As described hereinabove, In some embodiments, at least one of electrodes 12a, 12b are configured to perform sensing functionalities. For example, at least one of the electrodes may be configured to record the subject's ECG. Referring to FIG. 2B, In some embodiments, the wearable device includes one or more outward facing electrodes (i.e., the electrode is configured to face away from the subject's wrist, when the device is worn on the subject's wrist). In some embodiments, the wearable device is configured to record the subject's ECG by deploying an ECG sensor 16 using the outward-facing electrode as a first ECG electrode and using one of wrist-facing electrodes 12a, 12b as a second ECG electrode. Thus. by the user placing a finger or thumb on the outward-facing electrode 21, the wearable device is configured to detect the ECG signal between the subjects' finger or thumb, and the subject's wrist.

In some embodiments, wearable device 10 is configured to apply a neuromodulation treatment and/or to configure the parameters of a neuromodulation treatment based upon sensing and/or additional inputs as described in further detail hereinbelow. Alternatively or additionally, the wearable device is configured such that a user applies a treatment at regular intervals using the device, rather than the treatment being applied in response to sensing an episode or a potential episode. For example, and purely by way of illustration, the user may use the device for a treatment session lasting between 5 minutes and 1 hour at a rate of between once per day and once per month. In some embodiments, control module 18 and/or external computing devices 28 is configured such that it conditions the application of a treatment upon the user recording an ECG within a given time period (e.g., within one hour, 30 minutes, 10 minutes, or 5 minutes) of a treatment being applied. In some embodiments, a current treatment is conditioned upon an ECG having been recorded within the given time period before the current treatment. In some embodiments, a current treatment is conditioned upon an ECG having been recorded within the given time period after a previous treatment. Typically, by conditioning the application of the treatment upon such ECG recordings, the system facilitates monitoring the efficacy of the treatments by allowing monitoring of the effect of treatments on the subject's ECG (with such monitoring either being performed by a healthcare professional and/or being performed automatically by the system).

Reference is now made to FIGS. 2D-2E which are schematic illustration of module 15 that is configured to apply a neuromodulation treatment to a subject and/or to monitor a subject, in accordance with some applications of the present invention. Typically, module 15 is generally similar to module 15 as described hereinabove with reference to wearable device 10. In some embodiments, electrodes 12a, 12b, PPG sensor 11, accelerometer 13, ECG sensor 16, and/or control module 18 are housed in a module 15, with these components typically having generally similar functionalities to those described hereinabove. In some embodiments, electrodes 12a, 12b are disposed about a wrist-facing side 15a of module 15, and PPG sensor 11, accelerometer 13, ECG sensor 16 may be disposed on an opposite face of module 15, e.g., an outer-facing side 15b of module 15. In other embodiments, one or more of PPG sensor 11, accelerometer 13, ECG sensor 16 may also be disposed about the wrist-facing side 15a of module 15.

As noted above, although some of the sensing and/or neuromodulation techniques and the algorithms for use therewith are described herein with reference to wearable device 10, the scope of the present invention includes practicing any one of the sensing and/or neuromodulation techniques and the algorithms using one of, both of, and/or any suitable combination of wearable device 10 and module 15. For example, such techniques and algorithms may be practiced using electrodes and/or sensors that are placed in contact with the subject's body in the absence of a wearable device, and/or using a wearable device that is worn on a portion of the subject's body other than the subject's wrist. In some embodiments, one or more of the subject's median, ulnar, radial, tibial, peroneal, subcostal, intravertebral nerves, and/or a different nerve is stimulated. In some embodiments, module 15 is placed on a different portion of the subject's body, for example, the subject's chest (typically, with electrodes 12a, 12b contacting the subject's skin and electrode 21 facing away from the subject's skin).

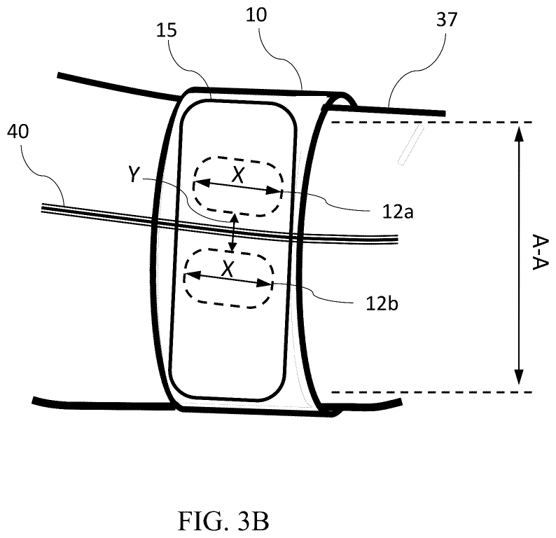

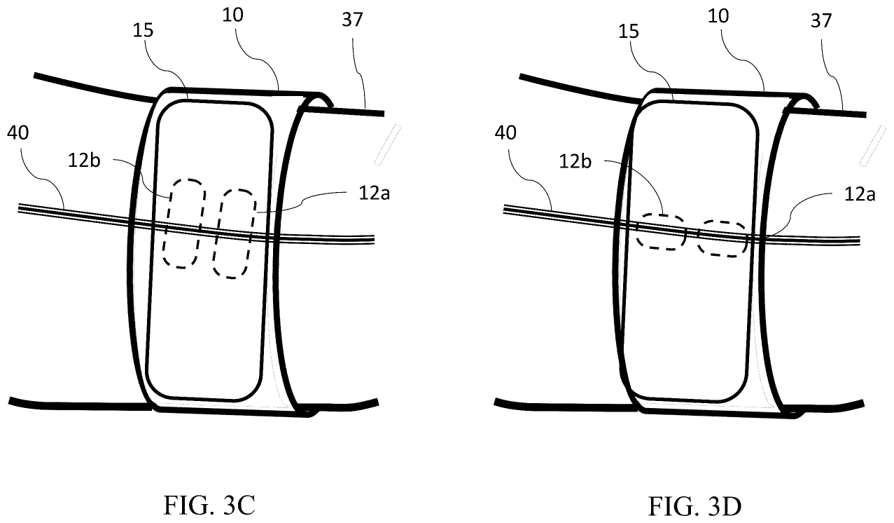

Reference is made to FIGS. 3A-3B. FIG. 3A is a schematic cross-sectional illustration of a human wrist/forearm 37, showing the location of the median and ulnar nerves in relation to the inner skin surface of wrist/forearm 37. FIG. 3A also shows a schematic exemplary module 15 of device 10 of the present disclosure, comprising two stimulation electrodes 12a, 12b disposed about a wrist-facing side 15a of module 15, as well as an ECG electrode 16 disposed on about an outer facing side 15b of module 15.

FIG. 3B is a schematic illustration of a human wrist/forearm 37 having a device 10 of the present disclosure wound and closed around it. Device 10 comprises module 15 positioned such that it faces an inner face of wrist/forearm 37. Module 15 comprises two stimulation electrodes 12a, 12b (each is exposed only on inner surface of wrist/forearm 37, and thus schematically depicted by a dashed elongated shape).

As can be seen in FIGS. 3A-3B, module 15 is disposed about wrist/forearm 37, such that stimulation electrodes 12a, 12b are positioned astride, e.g., one of either side of, median nerve 42, to make simultaneous dermal contact and to deliver the stimulation signal to median nerve 42. FIGS. 3C-3D show other alternatives for arranging electrodes 12a, 12b relative to median nerve 42, e.g., such that each of electrodes 12a, 12b is positioned across median nerve 42, either transversely (FIG. 3C) or lengthwise (FIG. 3D).

By way of background, targeting the median nerve represents an advantageous path for delivery of electrical stimulation signal aimed at reducing arrythmia-related events, for the following reasons:
The median nerve has a significantly larger cross-sectional area at the wrist than other upper-extremity nerves, such as the ulnar and radial nerves, and
The median is relatively close the skin, which increases electrical conduction of the electrical stimulation signal to the nerve.

Accordingly, in some embodiments, the present disclosure provides for a design of module 15 which is configured to ensure optimal positioning of module 15 and stimulation electrodes 12a, 12b relative to the median nerve 42, so as to maximize delivery of the stimulation signal to the median nerve 42.

In some embodiments, the same properties of median nerve 42 represent an advantageous path for measuring an ECG signal with respect to the subject, based on the positioning of module 15 and stimulation electrodes 12a, 12b relative to the median nerve 42, in conjunction with ECG sensor 16. Thus, in some embodiments, module 15 is configured to record the subject's ECG using ECG sensor 16 and one of electrodes 12a, 12b, by the subject placing a finger or thumb on the outward-facing ECG sensor 16, and thus recording an ECG signal between the subjects' finger or thumb and the subject's wrist.

In some embodiments, module 15 may define a rigid substantially planar body dimensioned to extend substantially across the span of the of the wrist/distal forearm 37 represented by the arrow A-A in FIGS. 3A-3B. In some embodiments, stimulation electrodes 12a, 12b may be disposed about a wrist-facing side 15a of module 15, along its lengthwise dimension, so as to be positioned in a specified position relative to median nerve 42 which straddles the pathway of median nerve 42. In some embodiments, electrodes 12a, 12b may be oriented about module 15 such that the lengthwise axis X of each electrode (shown in FIG. 3B) is disposed on either side of median nerve 42, proximately and substantially parallel to a longitudinal axis of the subject's median nerve 42.

Accordingly, in some embodiments, median nerve 42 may be electrically stimulated by closing an electrical conduction pathway between stimulation electrodes 12a, 12b and a skin region near or including a segment of median nerve 42.

In some embodiments, electrodes 12a, 12b may each have a width dimension which measures between 1-15 mm, e.g., 7 mm, and a length dimension which measures between 2-35 mm, e.g., 15 mm. In some embodiments, a distance Y (shown in FIG. 3B) between electrodes 12a, 12b may be in the range of between 1-10 mm, e.g., 4 mm.

In some embodiments, module 15 may be configured to be produced in a range of sizes and dimensions, each adapted to fit the anatomy and dimensions of wrists/forearms of various users, to ensure optimal placement of module 15 and stimulation electrodes 12a, 12b, and to maximize signal delivery to the median nerve. In some embodiments electrodes 12a, 12b may be square, oval or any other ordinary or unordinary shape, having sizes and surfaces areas lying in ranges similar to above-mentioned ranges.

With continued reference to FIGS. 3A-3B, in some embodiments, the present disclosure further provides for a technique for ensuring optimal positioning of module 15 (and thus, of stimulation electrodes 12a, 12b) about the inner surface of the wrist, to ensure maximal signal delivery to the median nerve. In some embodiments, the present technique is based on (i) measuring an electrical current with respect to an electrical conduction pathway between stimulation electrodes 12a, 12b, when device 10 is in use by a user and wound around the user's wrist, and (ii) comparing the measured current to a predetermined baseline current representing optimal positioning of electrodes 12a, 12b with respect to the subject. In some embodiments, when the measured electrical current is lower than the baseline value, device 10 of the present disclosure may issue an indication to the user and/or a healthcare practitioner to adjust the positioning of the device until the measured current is approaching or equal to the baseline value.

The present inventors have found that, because of the unique properties of the median nerve (i.e., large cross-section and relative proximity to skin surface), maximal electrical current values can be measured with respect to an electrical conduction pathway between stimulation electrodes 12a, 12b and a skin region near or including a segment of median nerve 42. Thus, with respect to a particular user, optimal positioning of electrodes 12a, 12b relative to median nerve 42 will represent a higher measured current value, as compared to all other possible positioning of electrodes 12a, 12b about the inner surface of the wrist. Table 1 below shows results of an experiment conducted by the present inventors with respect to three subjects. As can be seen in the results, for each of the subjects, maximal current measurement result is achieved when the electrodes 12a, 12b are in the optimum placement (M for ‘median’), over sub-optimal locations (R for ‘radial’ and/or U for ‘ulnar’ or U-Dist. For ‘Ulnar-Distal’). The values are given in milliamps (ma). Average current measurements represent at least 33 different measurements.

TABLE 1

| | Subject 1 | | | Subject 2 | | Subject 3 | | |
|---|---|---|---|---|---|---|---|---|
| Location | M | R | U | M | R | M | U-Dist. | U |
| Average | 5.367 | 3.918 | 2.288 | 4.473 | 2.970 | 5.258 | 1.520 | 3.708 |
| Current (ma) | | | | | | | | |
| p-Value | | <0.001 | <0.001 | | <0.001 | | <0.001 | <0.001 |

Accordingly, with continued reference to FIGS. 3A-3B, and with reference back to FIG. 1B, in some embodiments, the present disclosure provides for measuring a maximal baseline current value with respect to a subject, which represents an optimal positioning of electrodes 12a, 12b about an inner wrist skin area relative to the path of the median nerve. In some embodiments, when device 10 of the present disclosure is in use by the subject, placement module 22 (shown in FIG. 1B) may be configured to measure an electrical current with respect to an electrical conduction pathway between stimulation electrodes 12a, 12b, and compare the measured current to the predetermined baseline current. In some embodiments, when the measured electrical current is lower than the baseline value, placement module 22 may be configured to issue a suitable notification to the user and/or a healthcare practitioner to adjust the positioning of device 10. In some embodiments, placement module 22 may be configured to monitor device placement by performing current measurement upon user request, based on a planned scheduled, or periodically.

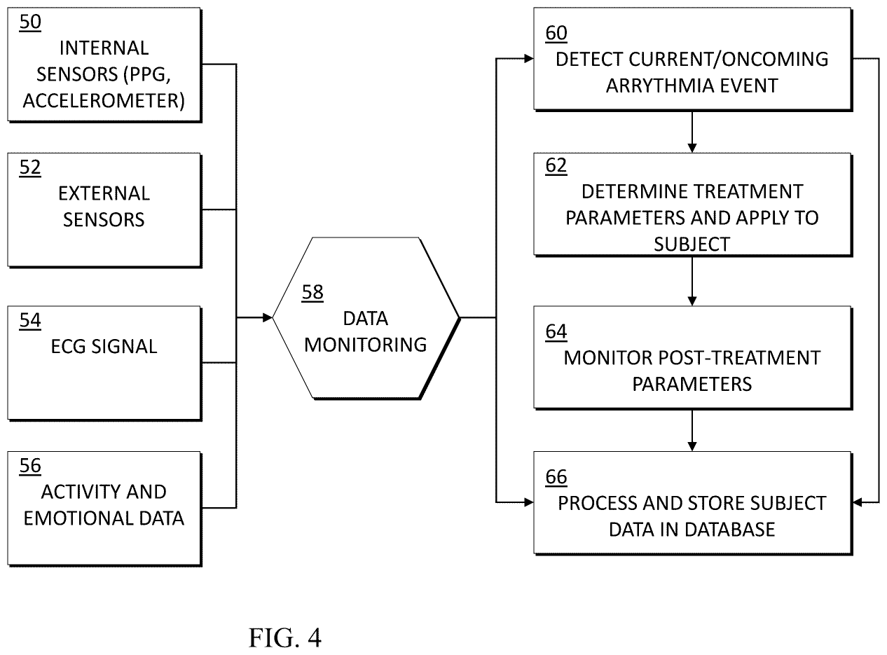

Reference is now made to FIG. 4 is a schematic diagram of the functional steps in a method for monitoring and treating a subject, in accordance with some applications of the present invention, using, e.g., the exemplary system described with reference to FIGS. 1A-1B and/or the exemplary devices described with reference to FIGS. 2A-2D and 3A-3B. As described hereinabove, In some embodiments, wearable device 10 includes one or more sensors in addition to electrodes 12a, 12b (which are configured to record the subject's ECG), such as PPG sensor 11 and/or accelerometer 13.

In some embodiments control module 18 and/or external computing devices 28 are configured to drive the wearable device to perform continuous, intermittent, and/or periodic monitoring, measurement and data acquisition with respect to a subject (step 58). For example, control module 18 and/or external computing devices 28 are configured to acquire data from internal sensors (step 50) within device 10, e.g., PPG measurements from the PPG sensor 11, data from accelerometer 13, and/or an ECG signal from ECG sensor 16. In some embodiments, step 58 may include round-the-clock monitoring and treatment. The monitoring may be continuous or scheduled, for example, every predetermined number of minutes or hours, as advised by a health care provider. In some embodiments the monitoring may be based on activity state of a subject as determined by the PPG/ECG sensors and accelerometer, for example monitoring may be initiated when subject is stationary as indicated by accelerometer data.

In some embodiments, control module 18 and/or external computing devices 28 are configured to analyze the acquired data (step 60), e.g., the PPG measurements, ECG signal, and/or the data received from the accelerometer, and to determine that (i) the subject may currently be undergoing arrythmia-related event, e.g., AF, PAC, AT or (ii) the subject is predicted to undergo an arrythmia-related event (e.g., in response to detecting a change in HRV, heart rate recovery, and/or heart rate). Typically, control module 18 and/or external computing devices 28 are configured to analyze data (step 60) acquired from the internal sensors (step 50), e.g., PPG sensor 11 and/or ECG sensor 16, to detect, e.g., heartrate-related episodes such as unexplained changes in heart rate, an unstable heart rate, measures of HRV, and/or a change in heart rate recovery, and to generate a prediction in response thereto, followed by a stimulation treatment for prevention purposes.

In some embodiments, control module 18 and/or external computing devices 28 are further configured to acquire additional and/or other data, including, but not limited to:
Data from one or more external sensors 23 (step 52), e.g., an acoustic sensor, optical sensor, a pressure sensor (e.g., configured to measure blood pressure), and/or a visual sensor;
an ECG signal with respect to the subject (step 54), using, e.g., ECG sensor 16 and/or an external ECG sensor;
one or more subject-related activity status, emotional status and or physiological data, including (step 56):
   current and/or recent activity status of the subject (e.g., eating, lying down, sleeping, walking, running, exercising, driving, etc.),
   a prandial status of the subject (e.g., hungry, currently eating, recently ate, etc.),
   an emotional state of the subject (e.g., stressed, relaxed, positive mood, depressed, anxious, traumatized, etc.), and/or
   additional physical symptoms (e.g., fatigue, tightness of the chest, palpitations, dizziness, fainting, head-ache, shortness of breath, sensation of “emptiness” in the chest, rapid or fluttering heartbeats, skipping heart beats, pressure in the throat, coldness or chills, dehydration,), and/or
   additional physiological parameters (e.g., blood-related parameters, anemia, digestion-related symptoms, insomnia, hormonal data).

In some embodiments, the following set of data may be obtained from the PPG sensor 11 and/or ECG sensor 16 and/or other heart-rate measurement in steps 50 and 54:
HRV: Heart rate variability,
RR interval: The time elapsed between two successive R-waves,
AVNN: Average interval between normal heart beats,
SDNN: The standard deviation of NN intervals,
rMSSD: root mean square of successive differences between normal heartbeats,
pNN50: The mean number of times an hour in which the change in successive normal sinus (NN) intervals exceeds 50 ms, and
LF and HF: Low and high frequency activity.

In some embodiments, subject-related parameters may be self-reported by the subject, e.g., via user interface 14. For example, the subject may be prompted to input information regarding their activity status, prandial status, emotional state, and/or physical symptoms. In some embodiments, the subject is prompted to input information regarding their lifestyle and/or activities, for example, via user interface 14. For example, the subject may be prompted to input information regarding their emotional state, physical symptoms, and/or prandial status, as detailed immediately above.

In some embodiments, control module 18 and/or external computing devices 28 are configured or prompt the subject to input one or more of these data, and/or to record an ECG for acquisition, e.g., sensor 16. Alternatively or additionally, the computing device prompts the subject to take additional measurements. Further alternatively or additionally, control module 18 drives one or more sensors to automatically detect additional physiological parameters of the subject. In some embodiments, control module 18 is configured to receive additional data from other sensors, such as acoustic sensors, pressure sensors, optical sensors, and/or visual sensors, which may be disposed within the wearable device or disposed externally of the wearable device.

In some embodiments, control module 18 and/or external computer processor 28 are configured to automatically derive one or more of the activity and/or emotional data (step 56). For example, control module 18 may derive a current activity status (e.g., eating, lying down, sleeping, walking, running, exercising, etc.) and/or a recent activity status of the subject, based upon data that are received from the accelerometer and/or the PPG sensor. Alternatively or additionally, control module 18 may derive a current emotional state (e.g., stressed, relaxed, etc.) and/or a recent emotional state of the subject, based upon data that are received from PPG sensor 11, accelerometer 13, and/or the ECG sensor 16.

In some embodiments, control module 18 is configured to receive the time of day and correlate this to measurements and/or other data as described in the present paragraph and the above paragraphs. In some embodiments, in this manner, control module 18 is configured to build a database of how the subject's activities, emotional states, prandial status, and/or physiological parameters typically vary over the course of a day. In some embodiments, control module 18 is configured to measure and/or receive an input that is indicative of the subject's hormonal state, symptoms (such as chest discomfort), blood-related parameters (e.g., an indication of anemia, B12 levels, folic acid levels, etc.). Accordingly, in some embodiments, control module 18 is configured to build a database which stores (step 64), e.g., in memory storage device 18a, the subject's activity status, emotional states, prandial status, and/or physiological parameters over time, e.g., over the course of an hour, a day, a week, a month, a year. In some embodiments, control module 18 is configured to measure and/or receive an input that is indicative of the subject's hormonal state, symptoms (such as chest discomfort, palpitations, skipping heart beats—as mentioned above), blood-related parameters (e.g., an indication of anemia, iron deficiency, ferritin deficiency B12 levels, folic acid levels, etc.).

In some embodiments, in step 60, control module 18 processes the received data, to determining and/or predict that the subject is currently undergoing an arrythmia-related event, e.g., AF, for example, based upon the subject's ECG signal and/or additional data. Alternatively or additionally, control module 18 is configured to predict an oncoming arrythmia-related event. For example, control module 18 may detect that a rate of increase in premature atrial contractions (PACs), which is interpreted as being a precursor to the subject potentially undergoing AF in the near future. Alternatively or additionally, control module 18 may predict an upcoming arrythmia-related event based upon data relating to the subject's current activity status, emotional state, and/or prandial status, in combination with additional physiological data. In some embodiments, control module 18 detects that the subject is currently undergoing an arrythmia episode (such as an occurrence of premature atrial contraction, premature ventricular contractions, supraventricular tachycardia, and/or atrial tachycardia). In some embodiments, control module 18 may be configured to determine an oncoming arrythmia-related event based on a history of known recurrence of an arrythmia-related event in the subject (e.g., daily at or about a specified time of the day).

In some embodiments, control module 18 is configured to detect an arrhythmia-related event as being one or more of AF, premature atrial complex (APC/PAC), premature ventricular complex (VPC/PVC), supraventricular tachycardia (SVT), atrial tachycardia (AT), atrial flutter, atrioventricular node re-entrant tachycardia (AVNRT), Paroxysmal supraventricular tachycardia (PSVT), atrioventricular re-entrant tachycardia, Wolff-Parkinson-White syndrome, ventricular tachycardia (VT), torsades de pointes (TdP), long QT syndrome, heart block, and/or sick sinus syndrome.

In some embodiments, in step 62, based on the results of the detection step 60, control module 18 is configured to determine one or more treatment parameters, which include treatment session parameters and stimulation signal parameters. In some embodiments, treatment parameters may include, but are not limited to:
Bodily site or specified nerve to be targeted in the treatment;
number of treatment sessions;
overall treatment session duration;
treatment session timing;
number and duration of on/off treatment session cycles;
overall signal duration;
signal waveform (e.g., sinusoidal, triangular, rectangular, and/or saw tooth waveform);
signal amplitude (e.g., maximum, minimum, mean, and/or average amplitude);
signal intensity (e.g., maximum, minimum, mean, and/or average intensity);
signal frequency;
signal pulse parameters when the stimulation signal comprises a series of pulses (e.g., pulse duration and/or inter-pulse time intervals); and/or
synchronization of the signal with one or more other signals and/or reference activities (e.g., cardiac activity of the subject).

In some embodiments, step 62 may involve application of an intermittent stimulation signal (i.e. a voltage/current signal), that is to say, a series of electric pulses (each electric pulse essentially being a brief duration stimulation signal). In some embodiments, the time intervals between successive electric pulses and the shape (i.e., the modulation or the waveform) of each pulse may be controllably varied. In some embodiments, the stimulation pulses may be biphasic, that is to say, that a positive voltage portion of a pulse is followed, substantially immediately, by a negative voltage portion of the pulse. In some embodiments, the area covered by the positive voltage (indicative of conducted charge) portion is substantially equal to the area covered by the negative portion, thereby preventing/minimizing occurrence of electrolysis (which may result from the flow of electric currents through bodily tissue (in the target body part) containing fluids) when the pulse width is sufficiently narrow (e.g. smaller than 1 msec).

In some embodiments, in step 62, after determining treatment parameters, control module 18 is configured to drive the wearable device 10 to deliver and apply the determined treatment to the subject. As described hereinabove, typically the treatment comprises delivering an electrical signal into the subject's median and/or ulnar nerve via wrist-facing electrodes 12a, 12b.

In some embodiments, control module 18 may be further configured to classify and/or correlate the current or predicted arrythmia-related event or episode as being correlated with a parasympathetic or sympathetic state in the subject. In some embodiments, control module 18 performs this classification based upon the data monitored and collected in step 58 received from the various sensors. Alternatively or additionally, control module 18 performs the classification and correlation based upon subject lifestyle-related data and/or subject activity-related data. For example, control module 18 may be configured to derive whether the subject's parasympathetic tone is high and/or their sympathetic tone is low (or vice versa) based upon the subject's current and/or recent emotional state, activity status, and/or prandial status.

In some embodiments, control module 18 is configured to derive whether the subject's parasympathetic tone is high and/or their sympathetic tone is low (or vice versa) based upon the subject's HRV, which is known to be indicative of a person's autonomic state. This having been said, while a subject is undergoing an arrythmia-related event episode, it is typically not possible to measure the subject's HRV in a reliable manner. Therefore, In some embodiments, in order to classify the episode, control module 18 is configured to analyze the HRV of the subject within a given time period (e.g., within between 30 minutes and 1 minute), prior to the onset of the episode. In some embodiments, similar techniques are performed, but using the subject's heart rate and/or heart rate recovery as an alternative to or in addition to the subject's HRV.

In some embodiments, control module 18 is configured to derive whether the subject's parasympathetic tone is high and/or their sympathetic tone is low (or vice versa) based upon the subject's ECG signal, for example, the shape of the ECG signal, and/or the regularity of beats in the ECG signal.

In some embodiments, control module 18 is configured to derive whether the subject's parasympathetic tone is high and/or their sympathetic tone is low (or vice versa) based upon parameters relating to premature atrial contractions and/or atrial tachycardia episodes that the subject is currently undergoing and/or has recently undergone. For example, control module 18 may determine whether the premature atrial contractions and/or atrial tachycardia episodes occurred during exercise (indicating that they are correlated with excess sympathetic tone), or during rest (indicating that they are correlated with excess sympathetic tone).

Table 2 below summarizes the various subject-related data points and parameters that are typically associated with a sympathetic or parasympathetic state:

TABLE 2

| Autonomous Nervous | |
|---|---|
| System Activation State | Subject-Related State |
| Sympathetic | During physical activity (exercise) |
| | Increase hear rate reserve (HRR) |
| | Decreased heart rate variability (HRV) |
| | During a state of stress |
| | Increased Heart Rate |
| Parasympathetic | Restful state |
| | Sleep |
| | Recovery state |
| | During or after meals |
| | Increased HRV |
| | Decreased Heart Rate |

In some embodiments, when it is determined that the detected arrythmia-related event is correlated with a parasympathetic state in the subject, control module 18 is configured to determine that at least the stimulation signal frequency will be set at a frequency of between 40-50 Hz (e.g., 40, 41, 42, 43, 44, 45, 46, 47, 48, 49, or 50 Hz).

In some embodiments, when it is determined that the detected arrythmia-related event is correlated with a sympathetic state (i.e., excess sympathetic tone, and/or reduced parasympathetic or vagal tone), control module 18 is configured to determine that at least the stimulation signal frequency will be set at a frequency of between 1-5 Hz (e.g., 1, 2, 3, 4, or 5 Hz).

Table 3 below summarizes the various detected oncoming or current arrythmia-related event, their precursors, the applicable signal parameters, and the applicable treatment regime.

TABLE 3

| Arrythmia- | Heart Activity | Signal | Arrhyth- | |
|---|---|---|---|---|
| Related | Signal/ | Fre- | mia | Treatment |
| Event | Parameter | quency | Burden | Timing |
| Atrial Fibrillation/ | Acute increase in | 40-50 Hz | N/A | Upon |
| Premature Atrial | PACs/AT burden | | | detection |
| Contractions | | | | |
| Atrial Fibrillation/ | Parasympathetic | 40-50 Hz | <5% | 2 times |
| Premature Atrial | State | | | per week |
| Contractions | | | | scheduled |
| | | | | treatment |
| Atrial Fibrillation/ | Parasympathetic | 40-50 Hz | 5%- | 3 times |
| Premature Atrial | State | | 10.6% | per week |
| Contractions | | | | scheduled |
| | | | | treatment |
| Atrial Fibrillation/ | Parasympathetic | 40-50 Hz | >10.6% | 5 times |
| Premature Atrial | State | | | per week |
| Contractions | | | | scheduled |
| | | | | treatment |
| Atrial Fibrillation/ | Sympathetic | 1-5 Hz | <5% | 2 times |
| Premature Atrial | State | | | per week |
| Contractions | | | | scheduled |
| | | | | treatment |
| | | | 5%- | 3 times |
| | | | 10.6% | per week |
| | | | | scheduled |
| | | | | treatment |
| | | | >10.6% | 5 times |
| | | | | per week |
| | | | | scheduled |
| | | | | treatment |
| Atrial Fibrillation/ | Recuring time of | 40-50 Hz | N/A | 1 hour |
| Premature Atrial | day/Lifestyle | | | before |
| Contractions/ | event | | | expected |
| Premature | (Parasympathetic | | | event time |
| Ventricular | State) | | | |
| Contractions | | | | |
| Atrial Fibrillation/ | Recuring time of | 1-5 Hz | N/A | 1 hour |
| Premature Atrial | day/Lifestyle | | | before |
| Contractions/ | event | | | expected |
| Premature | (Sympathetic | | | event time |
| Ventricular | State) | | | |
| Contractions | | | | |
| Premature | Parasympathetic | 40-50 Hz | N/A | 2 times |
| Ventricular | State | | | per week |
| Contractions | | | | scheduled |
| | | | | treatment |
| Premature | Sympathetic | 1-5 Hz | N/A | 2 times |
| Ventricular | State | | | per week |
| Contractions | | | | scheduled |
| | | | | treatment |
| Premature | Acute increase in | 40-50 Hz | N/A | Upon |
| Ventricular | VPC burden | | | detection |
| Contractions | | | | |

In some embodiments, in step 64, after the treatment has been applied, control module 18 is further configured to monitor and collect post-treatment data with regard to the subject. In some embodiments, the post-treatment data may include some or all of the data items monitored and collected in step 58.

In some embodiments, in step 64, control module 18 is configured to prompt the subject to record an ECG within a given time period after the treatment has been applied. Alternatively or additionally, control module 18 prompts the subject to take additional measurements. Further alternatively or additionally, control module 18 drives one or more sensors to automatically detect additional physiological parameters of the subject. In some embodiments, control module 18 is configured to receive additional data from other sensors, such as acoustic sensors, pressure sensors, optical sensors, and/or visual sensors, which may be disposed within the wearable device or disposed externally of the wearable device. In some embodiments, control module 18 is configured to derive a post-treatment activity status of the subject (e.g., eating, lying down, sleeping, walking, running, exercising, etc.), based upon data that are received from the accelerometer and/or the PPG sensor. Alternatively or additionally, control module 18 may derive a current post-treatment emotional state of the subject (e.g., stressed, relaxed, etc.), based upon data that are received from the accelerometer and/or the PPG sensor. In some embodiments, the subject is prompted to input information regarding their post-treatment lifestyle and/or activities, for example, via user interface 14. For example, the subject may be prompted to input information regarding their emotional state (e.g., stressed, relaxed, positive mood, bad mood, etc.), symptoms (e.g., tiredness, tightness of chest, palpitations), and/or prandial status (e.g., hungry, currently eating, recently ate, etc.). In some embodiments, control module 18 is configured to receive the time of day and correlate this to measurements and/or other data as described in the present paragraph and the above paragraphs.

In some embodiments, in step 66, control module 18 is configured to store the subject's self-reported data (e.g., the subject's ECG) to respective treatments, and to correlate these with subject characteristics, time of day, subject activity status, subject emotional states, subject prandial status, subject HRV, etc. The database may include data and derived correlations that are subject specific, as well as data and derived correlations relating to a set of subjects, and/or all subjects that have been treated using such a system. In some embodiments, the data include data relating to the state of the subject prior to the treatment having been applied (for example, frequency of occurrences of arrythmia events).

In some embodiments, such data may include, but is not limited to:
Time until AF recurrence,
time until premature atrial contraction recurrence,
general atrial/ventricular activity,
atrial fibrillation burden measured as total/percentage of time in AF versus in sinus rhythm in a defined measurement period or number of AF episodes in a predetermined time period,
average length of time in AF events,
longest AF event,
self-reporting of symptoms and activity, emotional and prandial status by the subject,
previous treatment parameters used for treatments,
times of treatments (during the day),
treatment duration,
electrical signal parameters,
subject personal parameters (e.g., age, gender, height, weight, average cardiac burden).

In some embodiments, clinical outcomes are analyzed based upon change of cardiac burden, time until AF recurrence, time until premature atrial contraction recurrence, and/or self-reporting of symptoms and activity, emotional and prandial status by the subject. In some embodiments, in order to determine a state of a subject and/or a clinical outcome, control module 18 is configured to determine the subject's cardiac burden, which is typically determined based upon (a) a number of episodes in a defined period of time, (b) the length of each episode within the defined period of time, (c) percentage of time in AF within the defined time period, and/or (d) the severity of reported symptoms.

Typically, the results of the analysis that is performed in step 64 are then used as an input in steps 60, 62, and/or 64. That is to say that when a subject is undergoing a clinical episode or is predicted to undergo an episode, then in step 60, in addition to analyzing the current data or recently-acquired subject related data, the responses of this subject and/or other subjects to previous treatments are accounted for. Similarly, in step 64, in order to determine which treatment parameters to apply to the subject, in addition to analyzing the current data or recently-acquired subject related data, the responses of this subject and/or other subjects to previous treatments are accounted for, e.g., using artificial intelligence and/or machine learning algorithms.

In some embodiments, in performing the steps of the flowcharts shown in FIG. 4, the system utilizes combinations of the following data types:
Objective subject physiological parameter, which typically include: heart rate, heart rate reserve, heart rate recovery, HRV, premature atrial contractions, ventricular premature contractions, AF, atrial tachycardia, movement, sleep, activity.
Subjective subject parameters, which typically include: subject symptoms, prandial status, exercise, hormonal state, stress level, hydration level.
Externally measured physiological parameters, which typically include: blood work (e.g., TNF, IL-6, CRP), blood pressure.
Electrical stimulation parameters: pulse wave profile, pulse frequency, pulse intensity, pulse intermittency, treatment duration, treatment regimen.
Treatment outcomes, which typically include: objective data (such as ECG, HRV, heart rate recovery, heart rate, PPG), and subjective data (such as subject reports of symptoms and states).

In some embodiments, the system incorporates additional data into the data analysis, such as a database of historical information relating to subjects that have been treated using the system (including their treatments, and treatment outcomes), animal studies, DRG, vagus, and/or stellate recordings, scientific literature, and/or clinical know-how.

With respect to the treatments described herein it is noted that, in some cases, these treatments are used as an adjunct therapy to traditional therapies (e.g., ablation). It is further noted that, for a given subject, the duration and/or frequency of treatments is typically reduced over time, as the system trains the subject's nervous system to self-modulate, and/or due to changes in the subject's condition, or additional parameters.

### Experimental Results

Reference is now made to FIGS. 5A-5D, which are graphs showing experimental results of stimulation treatments that were performed on AF subjects, in accordance with some applications of the present invention.

Referring to FIG. 5A, this graph shows AF recurrences of a cohort of 14 AF subjects who were treated using a treatment as described herein (using a signal having a frequency of 40-50 Hz). The AF recurrence rates of subjects who were treated using a treatment as described herein is indicated with the dashed line. For the sake of comparison, recurrence rates for subjects that received standard medical care after cardioversion, as is indicated with the solid line. The data indicated in the solid line were derived from the following sources:
Predictors of arrhythmia recurrence in subjects with lone AF. Europace (2008) 10, 9-14.
Early Recurrences of AF After Electrical Cardioversion. JACC Vol. 31, No. 1. January 1998:167-73.
Early or Delayed Cardioversion in Recent-Onset AF. N Engl J Med. 2019 Jul. 25; 381(4):386-387.
Prevention of AF after cardioversion: results of the PAFAC trial. European Heart Journal (2004) 25, 1385-1394.

For the dashed line shown in FIG. 5A, data were collected from June 2018 to September 2020 from two private clinics in the center of Israel and were analyzed as retrospective open label case registry. The cohort included 14 males and females, aged 18 or older, with symptomatic and ECG-documented, recent onset AF (AF), lasting less than 48 hours. Neuromodulation treatments were delivered weekly with a wearable device as described herein for 10 weeks. Each session was conducted for 20 minutes. Following the 10 week period, treatment frequency was reduced to one session every two weeks and then to once a month for the rest of the follow up period. The subjects were followed for a period of up to two years. During the follow up period, 3 subjects experienced symptomatic and ECG-documented AF recurrences. These occurred in weeks 8, 28, and 42 in three separate subjects. Total reoccurrence rates were 7.1% (1 of 14) at 6 months and 21.4% (3 of 14) at 1 year follow up. Life quality as assessed by AFEQT (AF Effect on Quality of life) questioners were filed by 13 participants and showed significant improvements scores in AF related symptoms (26.1%), daily activities (14.7%), mental concern (37%) and overall score (23.4%), as indicated in Table 4 below.

TABLE 4

| | | n | Before | After | % Change | p-Value |
|---|---|---|---|---|---|---|
| AFEQT | Total | 13 | 65.5 | 85.49 | 23.38 | <0.001 |
| Score | Symptom | | 66.24 | 89.67 | 26.13 | <0.001 |
| | Daily Activities | | 75.82 | 88.95 | 14.75 | 0.057 |
| | Concern | | 50.58 | 80.23 | 36.996 | <0.001 |

FIG. 5D shows ECG signals of a 65 year old male suffering from AF before application of a treatment as described herein (Panel A) and after application of a 25 minute treatment as described herein (Panel B). It may be observed that before the treatment, the subject suffers from multiple premature ventricular contractions (indicated by the circled portions of the ECG signal). After the treatment, the subject no longer undergoes premature ventricular contractions.

The present inventors have conducted an experiment consisting of a cohort of 48 patients who received a total of 136 neuromodulation treatments according to aspects of the present disclosure, as described hereinabove.

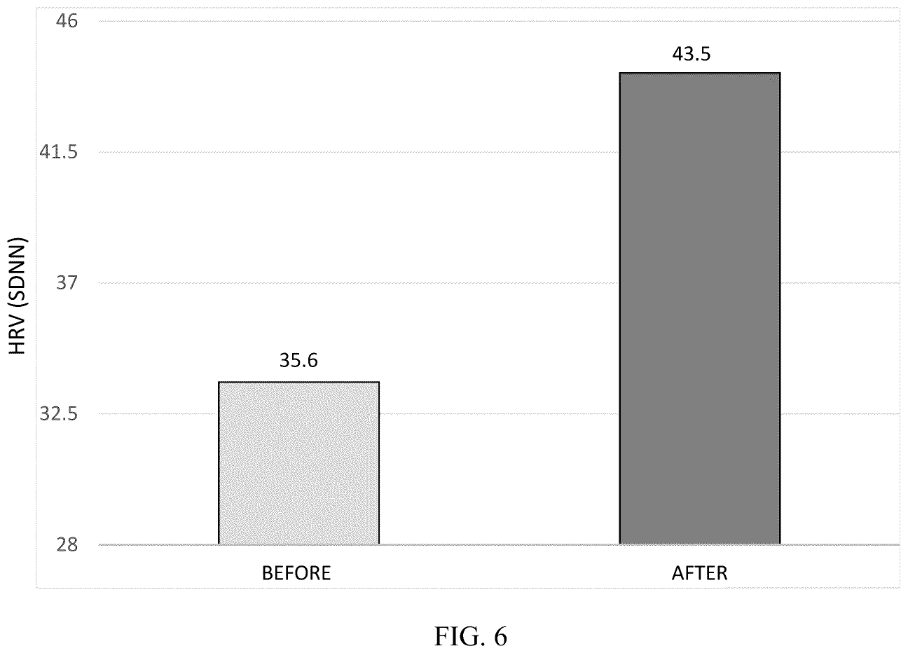

The patients demonstrated a low SDNN (standard deviation of NN intervals) of <36 ms, which is indicative of an increased risk of developing AF (see, e.g., Cardiac Autonomic Dysfunction and Incidence of Atrial Fibrillation: Results From 20 Years Follow-Up. J Am Coll Cardiol. 2017 Jan. 24; 69(3):291-299). The low SDNN was detected prior to providing the treatment, and was determined to indicate that that the autonomous nervous system of the subject is in a sympathetic state. Thus, the treatment included delivering to the subject a stimulation signal at a frequency of between 1-5 Hz. The results of the experiment are summarized in Table 5 below. FIG. 6 is a graph showing the results of the experiment. As can be seen, post treatment, the mean SDNN values for all patients increased by 22%.

TABLE 5

| Parameter | Mena Before | Mean After | % Change | p-Value |
|---|---|---|---|---|
| HR | 67.42 | 64.18 | 4.8 | <0.001 |
| SDNN(ms) | 35.65 | 43.50 | 22.02 | <0.001 |
| rMSSD(ms | 30.14 | 40.73 | 35.11 | <0.001 |
| LF | 301.58 | 394.64 | 30.86 | 0.014 |
| HF | 175.05 | 239.38 | 36.75 | <0.001 |
| VLF | 349.26 | 431.46 | 23.54 | 0.09 |
| Ln LF | 5.07 | 5.40 | 6.5 | <0.001 |
| LH/HF | 2.54 | 2.70 | 6.24 | 0.595 |

As described hereinabove, typically electrodes 12a, 12b are configured to be placed upon skin of a subject's wrist in a vicinity of the subject's ulnar nerve, and/or the subject's median nerve. Typically, an electrical stimulation signal is driven into the nerve via the electrodes, and the electrical stimulation signal configured to reduce occurrences of AF and/or arrhythmia events. In some embodiments, the electrical simulation signal has parameters which are as described hereinabove. The inventors of the present application have found that when the electrodes are properly positioned with respect to the nerve, and the stimulation parameters are set such as to generate the desired clinical outcome, the subject typically feels a sensation that starts in the vicinity of the electrodes and radiates along the nerve pathways (e.g., in the subject's wrist, hand, and/or fingers). Specifically, when the intensity of the electrical simulation signal is high enough to generate the desired clinical outcome, the subject typically feels a sensation that starts in the vicinity of the electrodes and radiates along the nerve pathways. However, it is typically undesirable for the electrical stimulation signal to elicit a motor response, which can occur if the intensity of the electrical stimulation signal is too high. For example, in some cases, the electrical stimulation signal can cause involuntary movement of a portion of the subject's body in the vicinity of the electrodes (e.g., in the subject's wrist, hand, and/or fingers). In view of the above, In some embodiments, control module and/or a different one of the computing devices 28 described herein is configured to receive an input from the subject indicating the reaction of the subject's body in the vicinity of the electrodes to the electrical stimulation signal. For example, control module 18 may receive inputs from the subject via user interface 14, and/or via smartphone 30, tablet device 32, and/or personal computer 34 (shown in FIG. 1A). Typically, control module 18 sets an intensity of the electrical stimulation signal, such that the electrical stimulation signal causes a sensation in a vicinity of the electrodes (e.g., a sensation that starts in the vicinity of the electrodes and radiates along the nerve pathways, as described above), but does not cause involuntary movement of a portion of the subject's body in the vicinity of the electrodes. For example, in response to receiving an input indicating that the electrical stimulation signal does not cause any sensation in a vicinity of the electrodes, control module 18 may increase an intensity of the electrical stimulation signal, and, in response to receiving an input indicating that the electrical stimulation signal causes involuntary movement of a portion of the subject's body in the vicinity of the electrodes, control module 18 may reduce an intensity of the electrical stimulation signal.

A further phenomenon that the inventors have found is that when the electrical simulation signal is applied at a given voltage, the current of the signal tends to be higher when the electrodes are properly placed with respect to the ulnar and/or the median nerve. It is noted that this is that case even when the electrodes are in good contact with the subject's skin. That is to say that, even when the electrodes are in good contact with the skin, the current is observed to vary and the current of the signal tends to be higher when the electrodes are properly placed with respect to the ulnar and/or the median nerve. This indicates that this phenomenon is unrelated to impedance that arises due to improper contact between the electrodes and the skin.

In order to treat AF and/or arrythmia conditions, it is desirable for the electrical stimulation signal to radiate along the ulnar and/or the median nerve, both of which are close to the surface of the skin. The inventors have shown that when the electrodes are properly placed with respect to the nerve, the current is higher due to the electrical stimulation signal radiating along the nerve, whereas when the electrodes are improperly placed with respect to the nerve, the current is lower. It is noted that a similar phenomenon is seen when the stimulation signal is applied at a given current. Namely, when the electrical simulation signal is applied at a given current, the voltage of the signal tends to be lower when the electrodes are properly placed with respect to the ulnar and/or the median nerve, even when the electrodes are in good contact with the subject's skin.

In view of the above, in some embodiments, control module 18 and/or a different one of the computing devices 28 described herein) is configured to drive the electrical stimulation signal into the ulnar and/or median nerve via the electrodes at a given voltage. control module 18 detects a current of the electrical stimulation signal, when the electrodes are in good contact with the skin of the subject's wrist, and in response to the current being below a threshold, control module 18 generates an output indicating that at least one of the electrodes should be moved (e.g., indicating that at least one of the electrodes should be moved closer to the selected nerve). For example, control module 18 may generate such outputs to the subject via user interface, and/or via smartphone 30, tablet device 32, and/or personal computer 34. In some embodiments, control module 18 and/or a different one of the computing devices described herein is configured to drive the electrical stimulation signal into the ulnar and/or median nerve via the electrodes at a given current. control module 18 detects a voltage of the electrical stimulation signal, when the electrodes are in good contact with the skin of the subject's wrist, and in response to the voltage being above a threshold, control module 18 generates an output indicating that at least one of the electrodes should be moved (e.g., indicating that at least one of the electrodes should be moved closer to the selected nerve). For example, control module 18 may generate such outputs to the subject via user interface 14, and/or via smartphone 30, tablet device 32, and/or personal computer 34.

As described hereinabove, In some embodiments, control module 18 and/or a different one of the computing devices described herein is configured to that the subject is currently undergoing AF, that the subject is currently undergoing an arrythmia episode, that the subject is predicted to undergo AF, and/or that the subject is predicted to undergo an arrythmia episode based upon analyzing the subject's ECG signal. In some embodiments, control module 18 is configured to classify an AF and/or an arrhythmia event as being one or more of premature atrial complex (APC/PAC), premature ventricular complex (VPC/PVC), supraventricular tachycardia (SVT), atrial tachycardia (AT), atrial flutter, atrioventricular node re-entrant tachycardia (AVNRT), Paroxysmal supraventricular tachycardia (PSVT), atrioventricular re-entrant tachycardia, Wolff-Parkinson-White syndrome, ventricular tachycardia (VT), Torsades de pointes, long QT syndrome, heart block, and/or sick sinus syndrome. In some embodiments, control module 18 is configured to derive whether the subject's parasympathetic tone is high and/or their sympathetic tone is low (or vice versa) based upon the subject's ECG signal.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, a field-programmable gate array (FPGA), or a programmable logic array (PLA) may execute the computer readable program instructions by utilizing state information of the computer readable program instructions to personalize the electronic circuitry, in order to perform aspects of the present invention. In some embodiments, electronic circuitry including, for example, an application-specific integrated circuit (ASIC), may be incorporate the computer readable program instructions already at time of fabrication, such that the ASIC is configured to execute these instructions without programming.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In the description and claims, each of the terms “substantially,” “essentially,” and forms thereof, when describing a numerical value, means up to a 20% deviation (namely, ±20%) from that value. Similarly, when such a term describes a numerical range, it means up to a 20% broader range—10% over that explicit range and 10% below it).

In the description, any given numerical range should be considered to have specifically disclosed all the possible subranges as well as individual numerical values within that range, such that each such subrange and individual numerical value constitutes an embodiment of the invention. This applies regardless of the breadth of the range. For example, description of a range of integers from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 4, and 6. Similarly, description of a range of fractions, for example from 0.6 to 1.1, should be considered to have specifically disclosed subranges such as from 0.6 to 0.9, from 0.7 to 1.1, from 0.9 to 1, from 0.8 to 0.9, from 0.6 to 1.1, from 1 to 1.1 etc., as well as individual numbers within that range, for example 0.7, 1, and 1.1.

The descriptions of the various embodiments of the present invention have been presented for purposes of illustration, but are not intended to be exhaustive or limited to the explicit descriptions. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise,” “include,” and “have,” as well as forms thereof, are not necessarily limited to members in a list with which the words may be associated.

Where there are inconsistencies between the description and any document incorporated by reference or otherwise relied upon, it is intended that the present description controls.

What is claimed is:

1. A system comprising:
a control module;
a neuro-stimulation unit operationally controlled by said control module and configured to generate an electrical stimulation signal at a frequency of between 40-50 Hz; and
at least two electrodes configured to be positioned in simultaneous dermal contact with an inner surface of the wrist of a subject, proximately to a median nerve of the subject, wherein each of said at least two electrodes is connected to said neuro-stimulation unit for delivering said generated electrical stimulation signal from said neuro-stimulation unit to said subject,
wherein said electrical stimulation signal is configured to apply a neuromodulation treatment to said subject, to reduce occurrences of an arrhythmia-related condition in said subject.

2. (canceled)

3. (canceled)

4. The system according to claim 1, wherein said control module is configured to predict a current or oncoming occurrence of an arrythmia-related condition with respect to said subject, based on received input data that is indicative of at least one parameter selected from the following categories of parameters: activity status parameters of the subject, prandial status parameters of the subject, and emotional state parameters of the subject, and to operate said neuro-stimulation unit to generate said electrical stimulation signal based, at least in part, on said predicting, wherein said generated electrical stimulation signal is delivered from said neuro-stimulation unit via said at least two electrodes to said subject.

5. The system according to claim 4, wherein said:
(i) activity status parameters of the subject indicate that the subject is at least one of: eating, lying down, sleeping, walking, running, exercising, or driving;
(ii) prandial status parameters of the subject indicate that the subject is at least one of: hungry, currently eating, or recently ate; and
(iii) emotional state parameters of the subject indicate that the subject is at least one of: stressed, relaxed, in a positive mood, depressed, anxious, or traumatized.

6. The system according to claim 4, wherein said control module is further configured to receive, as an additional input, data that is indicative of at least one additional parameter with respect to the subject, selected from the group consisting of: fatigue, tightness of the chest, palpitations, dizziness, fainting, headaches, shortness of breath, sensation of “emptiness” in the chest, rapid or fluttering heartbeats, skipping heart beats, pressure in the throat, coldness or chills, dehydration, blood-related parameters, anemia diagnosis, digestion-related symptoms, insomnia, and hormonal data, and wherein said predicting is further based, at least in part, on correlating, in said subject, at least one of said parameters comprising said additional input, with an occurrence of said arrythmia-related condition, wherein said correlating is based on current and historical information associated with occurrences of said arrythmia-related condition in said subject.

7. (canceled)

8. The system according to claim 1, wherein said control module is further configured to receive, as input, a heart activity signal of said subject, and wherein said control module is further configured to process said heart activity signal to derive one or more heart activity-related parameters selected from the group consisting of: heart rate variability (HRV), heart rate recovery, heart rate reserve, premature atrial contractions (PAC), ventricular premature contractions, atrial tachycardia, supraventricular tachycardia, RR interval, average interval between normal heart beats (AVNN), standard deviation of NN intervals (SDNN), root mean square of successive differences between normal heartbeats (rMSSD), pNN50, low frequency activity (LF), and high frequency activity (HF).

9. (canceled)

10. The system according to claim 8, wherein said control module is further configured to detect, based on said heart activity-related parameters, a current or oncoming occurrence of said arrythmia-related condition with respect to said subject, and to operate said neuro-stimulation unit to generate said electrical stimulation signal based, at least in part, on said detecting, and wherein said generated electrical stimulation signal is delivered from said neuro-stimulation unit via said at least two electrodes to said subject.

11. The system according to claim 1, wherein said control module is further configured to determine that the autonomous nervous system of said subject is in a parasympathetic state, and to operate said neuro-stimulation unit to generate said electrical stimulation signal based, at least in part, on said determining.

12. (canceled)

13. The system according to claim 1, further comprising at least one of: an electrocardiogram (ECG) sensor, a photoplethysmogram (PPG) sensor, and an accelerometer.

14. (canceled)

15. (canceled)

16. (canceled)

17. (canceled)

18. (canceled)

19. A method comprising:
providing a system comprising:
a control module,
a neuro-stimulation unit operationally controlled by said control module and configured to generate an electrical stimulation signal at a frequency of between 40-50 Hz, and
at least two electrodes configured to be positioned in simultaneous dermal contact with an inner surface of the wrist of a subject, proximately to a median nerve of the subject, wherein each of said at least two electrodes is connected to said neuro-stimulation unit for delivering said generated electrical stimulation signal from said neuro-stimulation unit to said subject,
wherein said electrical stimulation signal is configured to apply a neuromodulation treatment to said subject, to reduce occurrences of an arrhythmia-related condition in said subject; and
placing said at least two electrodes in simultaneous dermal contact with an inner surface of the wrist of said subject, such that each of said at least two electrodes is positioned along a lengthwise axis thereof, proximately, and substantially parallel to, a longitudinal axis of said median nerve.

20. (canceled)

21. (canceled)

22. The method according to claim 19, further comprising predicting a current or oncoming occurrence of an arrythmia-related condition with respect to said subject, based on received input data that is indicative of at least one parameter selected from the following categories of parameters: activity status parameters of the subject, prandial status parameters of the subject, and emotional state parameters of the subject; and operating said neuro-stimulation unit to generate said electrical stimulation signal based, at least in part, on said predicting, wherein said generated electrical stimulation signal is delivered from said neuro-stimulation unit via said at least two electrodes to said subject.

23. The method according to claim 22, wherein said control module is further configured to receive, as an additional input, data that is indicative of at least one additional parameter with respect to the subject, selected from the group consisting of: fatigue, tightness of the chest, palpitations, dizziness, fainting, headaches, shortness of breath, sensation of “emptiness” in the chest, rapid or fluttering heartbeats, skipping heart beats, pressure in the throat, coldness or chills, dehydration, blood-related parameters, anemia diagnosis, digestion-related symptoms, insomnia, and hormonal data, and wherein said predicting is further based, at least in part, on correlating, in said subject, at least one of said parameters comprising said additional input, with an occurrence of said arrythmia-related condition, wherein said correlating is based on current and historical information associated with occurrences of said arrythmia-related condition in said subject.

24. (canceled)

25. The method according to claim 19, wherein said control module is further configured to receive, as an additional input, a heart activity signal of said subject, and wherein said control module is further configured to process said heart activity signal to derive one or more heart activity-related parameters selected from the group consisting of: heart rate variability (HRV), heart rate recovery, heart rate reserve, premature atrial contractions (PAC), ventricular premature contractions, atrial tachycardia, supraventricular tachycardia, RR interval, average interval between normal heart beats (AVNN), standard deviation of NN intervals (SDNN), root mean square of successive differences between normal heartbeats (rMSSD), pNN50, low frequency activity (LF), and high frequency activity (HF).

26. (canceled)

27. The method according to claim 25, further comprising detecting, based on said one or more heart activity-related parameters, a current or oncoming occurrence of said arrythmia-related condition with respect to said subject; and operating said neuro-stimulation unit to generate said electrical stimulation signal based, at least in part, on said detecting, wherein said generated electrical stimulation signal is delivered from said neuro-stimulation unit via said at least two electrodes to said subject.

28. The method according to claim 19, wherein said control module is further configured to determine that the autonomous nervous system of said subject is in a sympathetic state or a parasympathetic state, and to operate said neuro-stimulation unit to generate said electrical stimulation signal based, at least in part, on said determining.

29. (canceled)

30. (canceled)

31. (canceled)

32. (canceled)

33. (canceled)

34. (canceled)

35. A system comprising:
a control module;
a neuro-stimulation unit operationally controlled by said control module and configured to generate an electrical stimulation signal; and
at least two electrodes configured to be positioned in simultaneous dermal contact with an inner surface of the wrist of a subject, wherein each of said at least two electrodes is connected to said neuro-stimulation unit for delivering said generated electrical stimulation signal from said neuro-stimulation unit to said subject,
wherein said control module is configured to:
receive, as input, a heart activity signal of said subject,
detect, based on said heart activity signal, a current or oncoming occurrence of an arrythmia-related condition with respect to said subject, and
operate said neuro-stimulation unit to generate said electrical stimulation signal at a frequency of between 40-50 Hz, wherein said generated electrical stimulation signal is delivered from said neuro-stimulation unit via said at least two electrodes to said subject, and
wherein said electrical stimulation signal is configured to apply a neuromodulation treatment to said subject, to reduce occurrences of said arrhythmia-related condition in said subject.

36. The system according to claim 35, wherein said control module is configured to detect said oncoming occurrence of said arrythmia-related condition, based, at least in part, on detecting, in said heart activity signal, at least one of: premature atrial complexes (PAC), and premature ventricular complexes (PVC), wherein said detecting is based on measuring, in said heart activity signal, a percentage increase in said one of PAC and PVC, relative to a baseline measurement in said subject.

37. (canceled)

38. (canceled)

39. The system according to claim 35, wherein said control module is further configured to process said heart activity signal to derive one or more heart activity-related parameters selected from the group consisting of: heart rate variability (HRV), heart rate recovery, heart rate reserve, premature atrial contractions (PAC), ventricular premature contractions, atrial tachycardia, supraventricular tachycardia, RR interval, average interval between normal heart beats (AVNN), standard deviation of NN intervals (SDNN), root mean square of successive differences between normal heartbeats (rMSSD), pNN50, low frequency activity (LF), and high frequency activity (HF), and wherein said detecting is based, at least in part, on said one or more heart activity-related parameters.

40. (canceled)

41. The system according to claim 35, wherein said control module is further configured to predict a current or oncoming occurrence of an arrythmia-related condition with respect to said subject, based on received input data that is indicative of at least one additional parameter selected from the group consisting of: an activity status of the subject, a prandial status of the subject, and an emotional state of the subject, and to operate said neuro-stimulation unit to generate said electrical stimulation signal based, at least in part, on said determining.

42. The system according to claim 35, wherein said control module is further configured to determine that the autonomous nervous system of said subject is in a parasympathetic state, and to operate said neuro-stimulation unit to generate said electrical stimulation signal based, at least in part, on said determining.

43. (canceled)

44. (canceled)

45. The system according to claim 35, wherein said arrhythmia-related condition is one or more of: atrial fibrillation (AF), premature atrial complex (PAC), premature ventricular complex (PVC), supraventricular tachycardia (SVT), atrial tachycardia (AT), atrial flutter, atrioventricular node re-entrant tachycardia (AVNRT), paroxysmal supraventricular tachycardia (PSVT), atrioventricular re-entrant tachycardia, Wolff-Parkinson-White syndrome, ventricular tachycardia (VT), torsades de pointes (TdP), long QT syndrome, heart block, and sick sinus syndrome.

46. (canceled)

47. (canceled)

48. (canceled)

49. (canceled)

50. (canceled)

51. (canceled)

52. (canceled)

53. (canceled)

54. (canceled)

55. (canceled)
